(19) United States
(12) Patent Application Publication
(10) Pub. No.: US 2010/0022635 A1
(43) Pub. Date: Jan. 28, 2010

(54) Heat shock protein 90 inhibitor dosing methods

(75) Inventor: Roger A. Rajewski, Lawrence, KS (US)

Correspondence Address: MERCHANT & GOULD PC, P.O. BOX 2903, MINNEAPOLIS, MN, 55402-0903, US

(73) Assignee: University of Kansas, Lawrence, KS (US)

(21) Appl. No.: 12/462,012

(22) Filed: Jul. 28, 2009

Related U.S. Application Data
(60) Provisional application No. 61/084,185, filed on Jul. 28, 2008.

(51) Int. Cl. A61K 31/352; A61P 35/00; A61P 3/10

(52) U.S. Cl. 514/457

(57) ABSTRACT

The disclosure provides novel dosing regimens for Hsp90 inhibitors for use in the treatment or prevention of a neurodegenerative disorder, an autoimmune disorder, or cancer. The methods involve administering one or more doses of a therapeutically effective amount of at least one Hsp90 inhibitor to a subject in need thereof such that no more than a single dose is administered within a period of about 7 days.

### CROSS-REFERENCE TO RELATED APPLICATIONS

This application claims the benefit of U.S. Provisional Application Ser. No. 61/084,185, filed on Jul. 28, 2008, which is hereby incorporated herein by reference in its entirety.

### STATEMENT REGARDING FEDERALLY SPONSORED RESEARCH OR DEVELOPMENT

Not applicable.

### BACKGROUND OF THE INVENTION

The 90 kDa heat shock proteins (“Hsp90”) belong to a family of chaperones that regulate intracellular functions and are required for the refolding of denatured proteins following heat shock, as well as the conformational maturation of a large number of key proteins involved in cellular processes. The Hsp90 family of chaperones is comprised of four different isoforms. Hsp90-alpha and Hsp90-beta are found predominately in the cytosol, the 94-kDa glucose-regulated protein (“GRP94”) is localized to the endoplasmic reticulum, and Hsp75/tumour necrosis factor receptor associated protein 1 (“TRAP-1”) resides mainly in the mitochondrial matrix. These Hsp90s bind to client proteins in the presence of cochaperones, immunophilins, and partner proteins to make the multiprotein complex responsible for conformational maturation of newly formed nascent peptides into biologically active three-dimensional structures.

Hsp90 is an ATP-dependent protein with an ATP binding site in the N-terminal region of the active homodimer. Disruption of the ATPase activity of Hsp90 results in the destabilization of multiprotein complexes and subsequent ubiquitination of the client protein, which undergoes proteasome-mediated hydrolysis. More specifically, in an ATP-dependent fashion, Hsp70 binds to newly synthesized proteins cotranslationally and/or posttranslationally to stabilize the nascent peptide by preventing aggregation. Stabilization of the Hsp70/polypeptide binary complex is dependent upon the binding of Hsp70 interacting protein (“HIP”), which occurs after Hsp70 binds to the newly formed peptide. Hsp70-Hsp90 organizing protein (“HOP”) contains highly conserved tetratricopeptide repeats (“TPRs”) that are recognized by both Hsp70 and Hsp90, promoting the union of Hsp70/HIP and Hsp90, which results in a heteroprotein complex. In the case of telomerase and steroid hormone receptors, the client protein is transferred from the Hsp70 system to the Hsp90 homodimer with concomitant release of Hsp70, HIP, and HOP. Upon binding of ATP and an immunophilin with cis/trans peptidyl prolyl-isomerase activity (FKBP51, FKBP52, or CyPA), the ensemble folds the client protein into its three-dimensional structure. In a subsequent event, p23 binds Hsp90 near the N-terminal region promoting the hydrolysis of ATP and release of the folded protein, Hsp90 partner proteins, and ADP.

Examples of proteins dependent upon Hsp90 for conformational maturation include oncogenic Src kinase, Raf, p185, mutant p53 (not normal p53), telomerase, steroid hormone receptors, polo-like kinase (“PLK”), protein kinase B (“AKT”), death domain kinase (“RIP”), MET kinase, focal adhesion kinase (“FAK”), aryl hydrocarbon receptor, RNA-dependent protein kinase (“PKR”), nitric oxide synthase (“NOS”), centrosomal proteins, and others. In addition, other proteins, such as cyclin dependent kinase 4 (“CDK4”), cyclin dependent kinase 6 (“CDK6”), and human epidermal growth factor receptor 2 (“Her-2”) are thought to be client proteins of Hsp90. Of these Hsp90 client proteins, Raf, PLK, RIP, AKT, FAK, telomerase, and MET kinase are directly associated with the six hallmarks of cancer: (1) self-sufficiency in growth signals; (2) insensitivity to antigrowth signals; (3) evasion of apoptosis; (4) unlimited replication potential; (5) sustained angiogenesis; and (6) tissue invasion/metastasis. Consequently, Hsp90 is a target for the development of cancer therapeutics because multiple signaling pathways can be simultaneously inhibited by disruption of the Hsp90 protein folding machinery.

Inhibitors of Hsp90 include the anti-tumor antibiotics geldanamycin (“GDA”), radicicol (“RDC”), herbimycin A (“HB”), a 17-allylamino derivative of GDA (“17-AAG”), and the synthetic ATP analog called PU3, as well as their chimeric derivatives. For a description of these compounds and others see Blagg et al., U.S. Pat. No. 7,210,630, incorporated herein by reference. Novobiocin, and its analogues, such as KU-1 and its 8-methyl derivative KU-32, are also Hsp90 inhibitors. The structure of novobiocin, KU-1, and KU-32 are provided below:

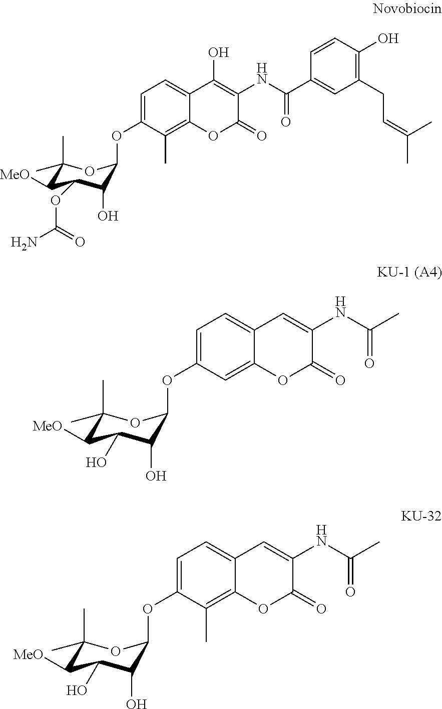

### BRIEF SUMMARY OF THE INVENTION

In one aspect, the disclosure provides a method for treating or preventing a medical condition selected from the group consisting of a neurodegenerative disorder (including but not limited to diabetic peripheral neuropathy), an autoimmune disorder (including but not limited to multiple sclerosis (MS)), and cancer, the method comprising administering according to a dosing regimen one or more doses of a therapeutically effective amount of at least one heat shock protein 90 inhibitor wherein the dosing regimen consists of no more than a single dose within a period of about 7 days. In one embodiment, the dosing regimen consists of a single dose. In another embodiment, the dosing regimen comprises at least two doses wherein the interval between consecutive doses is independently not less than about 7 days in length. In another embodiment, the dosing regimen comprises at least two doses wherein the interval between consecutive doses is independently from about 7 days to about 28 days in length. In another embodiment, the dosing regimen comprises at least two doses wherein the interval between consecutive doses is independently from about 14 days to about 28 days in length. In another embodiment, the dosing regimen comprises at least two doses wherein the interval between consecutive doses is independently from about 7 days to about 14 days in length. In another embodiment, the dosing regimen comprises at least two doses wherein the interval between consecutive doses is about 7 days in length. In another embodiment, the dosing regimen comprises at least two doses wherein the interval between consecutive doses is 7 days. In another embodiment, said dose comprises from about 2 mg/kg to about 20 mg/kg of said heat shock protein 90 inhibitor. In another embodiment, the heat shock protein 90 inhibitor is administered intravenously, intraperitoneally, or orally. In another embodiment, the Hsp90 inhibitor is:
N-(7-((2R,3R,4S,5R)-3,4-dihydroxy-5-methoxy-6,6-dimethyl-tetrahydro-2H-pyran-2-yloxy)-2-oxo-2H-chromen-3-yl)acetamide;
N-(7-((2R,3R,4S,5R)-3,4-dihydroxy-5-methoxy-6,6-dimethyltetrahydro-2H-pyran-2-yloxy)-8-methyl-2-oxo-2H-chromen-3-yl)acetamide;
N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)-1H-indole-2-carboxamide;
N-(7-((2R,3R,4S,5R)-3,4-Dihydroxy-5-methoxy-6,6-dimethyl-tetrahydro-2H-pyran-2-yloxy)quinolin-3-yl)-4-methoxy-3-(3-methoxyphenyl)-benzamide;
3-(3′,6-dimethoxybiphenyl-3-ylcarboxamido)-8-methyl-2-oxo-2H-chromen-7-yl propionate;
3-(3′,6-dimethoxybiphenyl-3-ylcarboxamido)-8-methyl-2-oxo-2H-chromen-7-yl cyclopropane carboxylate; or
3-(3′,6-dimethoxybiphenyl-3-ylcarboxamido)-6-methoxy-8-methyl-2-oxo-2H-chromen-7-yl acetate.

In another aspect, the disclosure provides a method for treating or preventing a medical condition selected from the group consisting of a neurodegenerative disorder, an autoimmune disorder, and cancer, the method comprising administering according to a dosing regimen n consecutive doses of a therapeutically effective amount of at least one heat shock protein 90 inhibitor to a subject in need thereof, where n is at least two. The dosing regimen thus comprises n-1 intervals between the n consecutive doses. Each of said n-1 intervals is independently not less than about 7 days in length.

In another aspect, the disclosure provides a method for treating or preventing a medical condition selected from the group consisting of multiple sclerosis and diabetic peripheral neuropathy. The method involves administering a plurality of consecutive doses of a therapeutically effective amount of at least one heat shock protein 90 inhibitor to a subject in need thereof. The interval between consecutive doses is independently from about 7 days to about 28 days in length and the Hsp90 inhibitor is selected from the group consisting of
N-(7-((2R,3R,4S,5R)-3,4-dihydroxy-5-methoxy-6,6-dimethyl-tetrahydro-2H-pyran-2-yloxy)-2-oxo-2H-chromen-3-yl)acetamide; and
N-(7-((2R,3R,4S,5R)-3,4-dihydroxy-5-methoxy-6,6-dimethyltetrahydro-2H-pyran-2-yloxy)-8-methyl-2-oxo-2H-chromen-3-yl)acetamide.

Additional aspects of the invention, together with the advantages and novel features appurtenant thereto, will be set forth in part in the description which follows, and in part will become apparent to those skilled in the art upon examination of the following, or may be learned from the practice of the invention. The objects and advantages of the invention may be realized and attained by means of the instrumentalities and combinations particularly pointed out in the appended claims.

### BRIEF DESCRIPTION OF THE DRAWINGS

FIG. 1 shows average mouse brain and plasma levels of KU-32 given to mice as a function of time post-administration.

FIG. 2 shows average mouse brain Hsp70 levels following a single IP injection of KU-32 (2 or 20 mg/kg).

FIG. 3 shows the average body weight of mice dosed with 2, 5, or 10 mg/kg of KU-32 by intraperatoneal injection on a daily basis in an EAE model.

FIG. 4 shows the average clinical score weight of mice dosed with 2, 5, or 10 mg/kg of KU-32 by intraperatoneal injection on a daily basis in an EAE model.

FIG. 5 shows the average clinical score of mice dosed with single vs. weekly injections of KU-32 in an EAE model.

FIG. 6 shows the average body weight of mice dosed with single vs. weekly injections of KU-32 in an EAE model.

FIG. 7 shows average nerve conduction velocity (NCV) measured in a mouse model of streptozotocin (STZ)-induced diabetic neuropathy after 12 weeks, prior to initiating KU-32 treatment.

FIG. 8 shows average mechanical hypoalgesia in a mouse model of streptozotocin (STZ)-induced diabetic neuropathy in KU-32 treated and untreated mice.

FIG. 9 shows average thermal hypoalgesia in a mouse model of streptozotocin (STZ)-induced diabetic neuropathy in KU-32 treated and untreated mice.

### DETAILED DESCRIPTION

The present disclosure is directed to methods for administering Hsp90 inhibitors. In particular, the present disclosure provides methods for the treatment and/or prevention of a medical condition selected from the group of cancer, autoimmune disorders, and neurodegenerative disorders. The methods involve administering according to dosing regimen at least one dose of a therapeutically effective amount of at least one heat shock protein 90 inhibitor to a subject in need thereof. Hereinafter, we refer to a dose of a therapeutically effective amount of at least one heat shock protein 90 inhibitor simply as a “dose.” Any Hsp90 inhibitor may be used in the methods disclosed herein. Non-limiting examples of Hsp90 inhibitors which may be used in the methods disclosed herein are described in detail below in the section entitled “Hsp90 Inhibitor Compounds.” Preferred Hsp90 inhibitors include N-(7-((2R,3R,4S,5R)-3,4-dihydroxy-5-methoxy-6,6-dimethyl-tetrahydro-2H-py-ran-2-yloxy)-2-oxo-2H-chromen-3-yl)acetamide (A4, KU-1) and N-(7-((2R,3R,4S,5R)-3,4-dihydroxy-5-methoxy-6,6-dimethyltetrahydro-2H-pyran-2-yloxy)-8-methyl-2-oxo-2H-chromen-3-yl)acetamide (KU-32). Preferably, each dose is administered in the form of a pharmaceutical composition. Suitable pharmaceutical compositions are described below in the section entitled “Pharmaceutical Compositions Used in the Dosing Methods.” The medical conditions which may be treated according to the disclosed methods are described in detail below in the section entitled “Medical Conditions.”

In one embodiment, a single dose is administered i.e. a dosing regimen consists of a single dose of a therapeutically effective amount of at least one heat shock protein 90 inhibitor. A physician may prescribe a single dose in order to treat a particular episode of a medical condition, even a chronic medical condition. If a single dose is administered according to a single dose dosing regimen, then preferably a subsequent single dose dosing regimen is not initiated sooner than about 7 days after the first dosing regimen. Thus, a physician may prescribe a single dose which is administed on Day 0 in order to treat a particular episode of a medical condition. If the physician decides to prescribe another single dose, preferably that further single dose is not administered before Day 7.

In another embodiment, two or more doses are administered i.e. a dosing regimen comprises at least two doses. Where two or more doses are administered, the interval between consecutive doses (i.e., the period of time between any one dose and the immediately following dose) is not less than about 7 days (e.g., 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 etc. days between consecutive doses). In general, if a dosing regimen includes n consecutive doses, there are n-1 intervals between those doses, and each of those n-1 intervals is independently not less than about 7 days. For example, the interval between consecutive doses may be from about 7 days to about 12 months. By way of another example, the interval between consecutive doses may be from about 7 days to about 6 months. By way of another example, the interval between consecutive doses may be from about 7 days to about 3 months. By way of another example, the interval between consecutive doses may be from about 7 days to about 2 months. By way of further example, the interval between consecutive doses may be from about 7 days to about 28 days (e.g., 7, 8, 9, 10, 11, 12, 13, 14, 15, 16, 17, 18, 19, 20, 21, 22, 23, 24, 25, 26, 27, or 28 days between one dose and the immediately following dose). In another example, the interval between doses is between about 7 days and about 14 days. In another example, the interval between doses is between about 14 days and about 28 days. In another example, the interval between doses is about 14 days. In another example, the interval between doses is about 28 days. In another example, the interval between doses is about 10 days. In another example, the interval between doses is about 7 days.

As used herein, the term “about” when referring to a number of days includes the specified number of days ±2 days. As used herein, the term “about” when referring to a number of months includes the specified number of months ±1 month.

An interval of about 7 to about 28 days is used as a non-limiting example for the following. If a dosing regimen consists of two doses there is a single interval between the doses which may be from about 7 days to about 28 days. If a dosing regimen consists of three doses, there are two intervals (the interval between the first dose and the second dose, and the interval between the second dose and the third dose) each of which is independently from about 7 days to about 28 days in length. If a dosing regimen consists of four doses, there are three intervals (the interval between the first dose and the second dose, the interval between the second dose and the third dose, and the interval between the third dose and the fourth dose) each of which is independently from about 7 days to about 28 days in length. Where the interval is from about 7 days to about 28 days in length, a dosing regimen consisting of n consecutive doses includes n-1 intervals between consecutive doses, each of which is independently about 7 to about 28 days in length.

In some embodiments, each of the intervals is the same length. For example, administration of a dose every 7 days (i.e. for the first time in the dosing regimen on Day 0, then on Day 7, then on Day 14, then on Day 21, and so on every 7 days until administration is no longer required) is an example of a dosing regimen where the intervals between consecutive doses are the same. By way of another example, the intervals are the same length if the doses are administered every 14 days i.e. for the first time in the dosing regimen on Day 0, then on Day 14, then on Day 28, then on Day 42, and so on until administration is no longer required. In other embodiments, at least one of the intervals between consecutive doses in a dosing regimen is different from at least one other interval in the dosing regimen. For example, the intervals are different lengths if the first three doses are administered every 7 days (i.e. Day 0, Day 7, Day 14) and then doses may be administered every 14 days after the third dose (i.e. on Day 28, Day 56, Day 84 and so on) until administration is no longer required. A dosing regimen where doses are administered on Day 0, Day 7, Day 14, and Day 28 is another example of a dosing regimen where the intervals between consecutive doses are different (with the interval between the dose at Day 0 and Day 7 being 7 days; the interval between the dose at Day 7 and Day 14 being 7 days; and the interval between the dose at Day 14 and Day 28 being 14 days). By way of another example, doses are administered on Day 0, Day 7, Day 15, Day 24, Day, 34, Day 35 and so on until administration is no longer required. In this example, the intervals between doses increases in increments of one day and thus no two intervals are the same. A physician may increase the interval between doses or decrease the interval between doses in view of the response of the subject to the Hsp90 inhibitor. Doses may be administered according to the instant dosing regimen for any length of time between about 1 week and a plurality of years.

As disclosed above, a “dose” comprises a therapeutically effective amount of a Hsp90 inhibitor(s). A “therapeutically effective amount” of an Hsp90 inhibitor is an amount which is effective for the treatment or prevention of the specified medical condition when administered according to the abovementioned dosing regimen. A dose for a single administration dosing regimen (i.e., where the dosing regiment comprises a single dose) may differ from each dose administered when a dosing regimen comprises two or more doses. For example, a dose for a single administration dosing regimen may comprise a higher amount of the Hsp90 inhibitor(s) than each dose in a dosing regimen that comprises two or more doses. “Treatment” of a medical condition includes the total or partial inhibition of the progression of the condition in a human or animal with at least a tentative diagnosis of the condition. “Treatment” of a medical condition also includes the total or partial alleviation of one or more symptoms of the medical condition in a human or animal with at least a tentative diagnosis of the condition. “Prevention” of a condition refers to stopping the development of the condition in a subject who has not been diagnosed as possibly having the condition, but who would normally be expected to develop the condition or be at increased risk for the condition due to age, familial history, genetic or chromosomal abnormalities, and/or due to the presence of one or more biological markers for the disorder or disease. “Prevention” refers also to a slowing of the development of the condition or to a delay in the onset of the condition.

The amount of a Hsp90 inhibitor(s) that is therapeutically effective will not only depend upon the dosing regimen, but also on the patient's size and gender, the condition to be treated, the severity of the condition and the result sought. For a given patient and condition, a therapeutically effective amount can be determined by methods known to those of skill in the art. For example, where the condition is cancer, a therapeutically effective amount refers to that amount of an Hsp90 inhibitor(s) which has the effect of (1) reducing the size of the tumor, (2) inhibiting (that is, slowing to some extent, preferably stopping) tumor metastasis, (3) inhibiting to some extent (that is, slowing to some extent, preferably stopping) tumor growth, and/or, (4) relieving to some extent (or, preferably, eliminating) one or more symptoms associated with the cancer.

In one embodiment, a therapeutically effective amount of a Hsp90 inhibitor may be between about 0.001 mg/kg and about 1,000 mg/kg, preferably between about 0.01 mg/kg and about 100 mg/kg, more preferably between about 2 mg/kg and about 20 mg/kg. The term “about” when used to refer to a therapeutically effective amount of a Hsp90 inhibitor expressed in mg/kg units includes the specified amount ±10%.

The “patient” or “subject” to be treated according to the dosing methods of the present invention can be any animal, e.g., dogs, cats, mice, monkeys, rats, rabbits, horses, cows, guinea pigs, sheep, and is preferably a mammal, such as a domesticated animal or a livestock animal. In another aspect, the patient is a human.

The dosing regimen of the Hsp90 inhibitor(s) provided by the disclosure is significantly different from traditional daily or multiple times per day dosing of other pharmaceuticals. Without being bound by theory or mechanism, it is believed that the pharmacological effect of Hsp90 inhibitors is not a result of the direct inhibition of Hsp90but is instead a result of the downstream processes that may be pharmacologically important long after the Hsp90 inhibitor levels have fallen below measurable levels. In particular, and without being bound by theory or mechanism, it is believed that the pharmacological effect of an Hsp90 inhibitor results from the transcriptional or translational up-regulation of heat shock protein genes, including the genes encoding Hsp70 and Hsp90. Traditional dosing (e.g. daily dosing) leads to the persistance of levels of the Hsp90 inhibitor sufficient to directly inhibit the newly-produced Hsp90 or Hsp70. Thus, dosing of the Hsp90 inhibitor on traditional temporal scales (e.g., daily) may actually inhibit any pharmacological benefit. Without being bound by theory or mechanism, it is believed that the dosing regimen of the instant disclosure optimizes the time between the decrease in the Hsp90 inhibitor to non-effective levels following administration, and the time that the heat shock protein levels are increased above basal levels. Thus, in still another aspect, the compounds of the present invention exhibit their therapeutic effects by upregulation of Hsp70 and/or Hsp90. Optimized dosing frequency where Hsp90 inhibitor levels are allowed to fall below effective levels while heat shock protein up-regulation to levels above basal may have a significant impact on the treatment of the following medical conditions which result, at least in part, from the presence of aberrant protein at the cellular level.

### Medical Conditions

As disclosed above, the dosing methods of the instant disclosure are useful for the treatment and/or prevention of a medical condition selected from the group of cancer, autoimmune disorders, and neurodegenerative disorders.

In one aspect, the condition treated and/or prevented by the methods of the disclosure is a neurodegenerative disorder. The term “neurodegenerative disorder” embraces a disorder in which progressive loss of neurons occurs either in the peripheral nervous system or in the central nervous system. Without being bound by theory, it is believed that the dosing regimens of the present disclosure enhance the neuroprotective effects of the Hsp90 inhibitor(s) during the treatment of the neurodegenerative disorder by inhibiting the progressive deterioration of neurons that leads to cell death. Examples of neurodegenerative disorders include, but are not limited to chronic neurodegenerative diseases such as diabetic peripheral neuropathy (including third nerve palsy, mononeuropathy, mononeuropathy multiplex,diabetic amyotrophy, autonomic neuropathy and thoracoabdominal neuropathy), Alzheimer's disease, age-related memory loss, senility, age-related dementia, Pick's disease, diffuse Lewy body disease, progressive supranuclear palsy (Steel-Richardson syndrome), multisystem degeneration (Shy-Drager syndrome), motor neuron diseases including amyotrophic lateral sclerosis (“ALS”), degenerative ataxias, cortical basal degeneration, ALS-Parkinson's-Dementia complex of Guam, subacute sclerosing panencephalitis, Huntington's disease, Parkinson's disease, multiple sclerosis (“MS”), synucleinopathies, primary progressive aphasia, striatonigral degeneration, Machado-Joseph disease/spinocerebellar ataxia type 3 and olivopontocerebellar degenerations, Gilles De La Tourette's disease, bulbar and pseudobulbar palsy, spinal and spinobulbar muscular atrophy (Kennedy's disease), primary lateral sclerosis, familial spastic paraplegia, Wernicke-Korsakoffs related dementia (alcohol induced dementia), Werdnig-Hoffmann disease, Kugelberg-Welander disease, Tay-Sach's disease, Sandhoff disease, familial spastic disease, Wohifart-Kugelberg-Welander disease, spastic paraparesis, progressive multifocal leukoencephalopathy, and prion diseases (including Creutzfeldt-Jakob, Gerstmann-Straussler-Scheinker disease, Kuru and fatal familial insomnia). Other conditions also included within the methods of the present invention include age-related dementia and other dementias, and conditions with memory loss including vascular dementia, diffuse white matter disease (Binswanger's disease), dementia of endocrine or metabolic origin, dementia of head trauma and diffuse brain damage, dementia pugilistica, and frontal lobe dementia. Also other neurodegenerative disorders resulting from cerebral ischemia or infarction including embolic occlusion and thrombotic occlusion as well as intracranial hemorrhage of any type (including, but not limited to, epidural, subdural, subarachnoid, and intracerebral), and intracranial and intravertebral lesions (including, but not limited to, contusion, penetration, shear, compression, and laceration). Thus, the term also encompasses acute neurodegenerative disorders such as those involving stroke, traumatic brain injury, schizophrenia, peripheral nerve damage, hypoglycemia, spinal cord injury, epilepsy, and anoxia and hypoxia.

In some embodiments, the neurodegenerative disorder is amyloidosis. Amyloidosis is observed in Alzheimer's Disease, hereditary cerebral angiopathy, nonneuropathic hereditary amyloid, Down's syndrome, macroglobulinemia, secondary familial Mediterranean fever, Muckle-Wells syndrome, multiple myeloma, pancreatic- and cardiac-related amyloidosis, chronic hemodialysis arthropathy, and Finnish and Iowa amyloidosis.

In preferred embodiments, the neurodegenerative disorder treated and/or prevented using the methods and compositions of the disclosure is diabetic peripheral neuropathy. In one such embodiment, a single dose is administered to a subject in need thereof. In another such embodiment, two or more doses are administered to a subject in need thereof such the interval between a dose and the immediately following dose is from about 7 days to about 28 days, more preferably from about 7 days to about 14 days. The Examples section of the instant disclosure includes examples of the treatment of this condition using the disclosed dosing regimen.

In another aspect, the condition treated and/or prevented by the methods of the disclosure is an autoimmune disorder. The term “autoimmune disorder” is intended to include disorders in which the immune system of a subject reacts to autoantigens, such that significant tissue or cell destruction occurs in the subject. The term “autoantigen” is intended to include any antigen of a subject that is recognized by the immune system of the subject. Autoimmune disorders include but are not limited to acute disseminated encephalomyelitis, Addison's disease, Alopecia universalis, ankylosing spondylitisis, antiphospholipid antibody syndrome, aplastic anemia, autoimmune hepatitis autoimmune infertility, autoimmune thyroiditis, autoimmune neutropenia, Behçet's disease, bullous pemphigoid, Chagas' disease, cirrhosis, Coeliac disease, Crohn's disease, Chronic fatigue syndrome, chronic active hepatitis, dense deposit disease, discoid lupus, dermatitis, luten-sensitive enteropathy, dysautonomia, endometriosis, glomerulonephritis, Goodpasture's disease, Graves' disease, Guillain-Barre syndrome, Hashimoto's disease, Hidradenitis suppurativa, idiopathic thrombocytopenia purpura, insulin dependent diabetes mellitus, interstitial cystitis, mixed connective tissue disease, multiple sclerosis (MS), myasthenia gravis, neuromyotonia, opsoclonus myoclonus syndrome, optic neuritis, Ord's thyroiditis, pemphigus vulgaris, pernicious anemia, polyarthritis, polymyositis, primary biliary cirrhosis, psoriasis, Reiter's syndrome, rheumatoid arthritis, sarcoidosis, scleroderma, Sjogren's syndrome, systemic lupus erythematosus, Takayasu's arteritis, temporal arteritis, ulcerative colitis, vitiligo, vulvodynia, warm autoimmune hemolytic anemia, or Wegener's granulomatosis. In a preferred aspect, the autoimmune disorder is multiple sclerosis or its animal model system termed experimental autoimmune encephalomyelitis (“EAE”).

In one aspect, the autoimmune disorder is mediated by a heat shock response which increases expresses expression of nitric oxide synthase, cytokines, and chemokines. Administration of the Hsp90 inhibitors of the present invention leads to a heat shock response due to the dissociation of HSF-1 from Hsp90.

In a preferred series of embodiments, the autoimmune disorder treated and/or prevented used in the methods and compositions of the disclosure is multiple sclerosis. In one such embodiment, a single dose is administered to a subject in need thereof. In another such embodiment, two or more doses are administered to a subject in need thereof such the interval between a dose and the immediately following dose is from about 7 days to about 28 days, more preferably from about 7 days to about 14 days.

In another aspect, the condition treated and/or prevented by the methods of the disclosure is cancer. Cancers which may be treated and/or prevented using the methods of the disclosure include breast cancer, colon cancer, pancreatic cancer, prostate cancer, head and neck cancer, lung cancer, gynecological cancers (including ovarian cancer and uterine cancer), brain cancer, germ cell cancer, urothelial cancer, esophageal cancer, and bladder cancer. In one embodiment of this aspect, cancer is treated and/or prevented by administering a single dose of the Hsp90 inhibitor(s) is administered to a subject in need thereof. In another such embodiment, two or more doses of the Hsp90 inhibitor(s) are administered to a subject in need thereof such the interval between a dose and the immediately following dose is from about 7 days to about 28 days, more preferably from about 7 days to about 14 days.

### Hsp90 Inhibitor Compounds

The present disclosure is directed to novel dosing strategies for Hsp90 inhibitors. Any Hsp90 inhibitor compound known in the art may be administered using the dosing methods disclosed herein. Suitable Hsp90 inhibitors include those which bind to either the Hsp90 N-terminal or C-terminal binding site. Examples of Hsp90 inhibitors to be utilized in the methods of the disclosure include the ansamycins, for example, geldanamycin, 17-allylamino-17-demethoxygeldanamycin (17-AAG, Tanespimycin, Retaspimycin hydrochloride), and 17-(Dimethylaminoethylamino)-17-demethoxygeldanamycin (17-DMAG); the macrolides, for example, Radicicol, Pochonin, and Radester; the purine-scaffold derivatives, for example, PU-3, PUFCl, and 8-arylsulfanyl adenine derivatives such as 8-(2-iodo-5-methoxy-phenylsulfanyl)-9-pent-4-ynyl-9H-purin-6-ylamine; pyrazoles such as CCT018159 (4-[4-(2,3-Dihydro-1,4-benzodioxin-6-yl)-5-methyl-1H-pyrazol-3-yl]-6-ethyl-1,3-benzenediol); and other known Hsp90 inhibitors such as Herbimycin, Shepardin, Cisplatin, Novobiocin; as well as the HADC inhibitors FR901228 (FK228, Romidepsin), LAQ824, LBH589 ((2E)-N-hydroxy-3-(4-(((2-(2-methyl-1H-indol-3-yl)ethyl)amino)methyl)phenyl)-2-Propenamide, Panobinostat) and trichostatin (TSA).

In one embodiment, the disclosure provides methods utilizing Hsp90 inhibitors which are analogues of novobiocin. Exemplary Hsp90 inhibitors which are analogues of novobiocin are described in (1) U.S. patent application Ser. No. 12/390,175 filed on Feb. 20, 2009, which was published Jun. 25, 2009 as Pub. No. US 2009/0163709; (2) U.S. patent application Ser. No. 12/390,011, filed on Feb. 20, 2009, which was published Jul. 23, 2009 as Pub. No. US 2009/0187014; (3) U.S. patent application Ser. No. 11/801,473, filed on May 10, 2007, which was published Nov. 22, 2007 as Pub. No. US 2007/0270452; and (4) U.S. patent application Ser. No. 11/266,149 filed on Nov. 3, 2005, which was published Sep. 7, 2006 as Pub. No. US 2006/0199776; each of which is incorporated herein by reference in its entirety. Other exemplary Hsp90 inhibitors are set forth in Blagg et al., U.S. Pat. No. 7,208,630, which is incorporated herein by reference.

In one embodiment, the disclosure provides methods utilizing an Hsp90 inhibitor compound of Formula I:

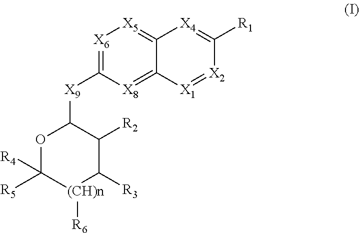

wherein R¹ is hydrogen, alkyl, alkenyl, alkynyl, carbocylic, heterocyclic, aryl, aralkyl, carboxyl, amido, amino, sulfanyl, sulfenyl, sulfonyl, or ether; or R¹ together with X² and the atom to which R¹ is attached form a heterocyclic ring having 4 to 8 ring members with at least one heteroatom selected from oxygen or nitrogen; or R¹ together with X⁴ and the atom to which R¹ is attached form a heterocyclic ring having 4 to 8 ring members with at least one heteroatom selected from oxygen or nitrogen;

wherein R² is hydrogen, hydroxy, or —R⁸—OR⁹, wherein R⁸ is a covalent bond or alkyl, and R⁹ is C-amido or acyl; or R² together with R³ and the atoms to which they are attached form a heterocyclic ring having 4 to 8 ring members with at least one heteroatom selected from oxygen or nitrogen;

wherein R³ is hydrogen, hydroxy, or —R¹⁰—O—R¹¹, wherein R¹⁰ is a covalent bond or alkyl, and R¹¹ is C-amido or acyl; or R³ together with R² and the atoms to which they are attached form a heterocyclic ring having 4 to 8 ring members with at least one heteroatom selected from oxygen or nitrogen;

wherein R⁴ is hydrogen, alkyl, hydroxy, carboxyl, —R¹²—O—R¹³, or —R¹²—R¹⁴,

wherein R¹² is a covalent bond or alkyl, and R¹³ is C-amido or acyl, and R¹⁴ is N-amido, —POR¹⁵R¹⁶, —SO₂R¹⁷, or sulfonamido and wherein R¹⁵, R¹⁶, R¹⁷ are independently alkoxy;

wherein R⁵ is hydrogen, alkyl, alkenyl, alkynyl, aryl, or aralkyl;

wherein R⁶ is hydrogen, alkyl, alkenyl, alkynyl, aryl, aralkyl, alkoxy, aryloxy, or aralkoxy;

wherein X₁ is —O—, —CO—, or —N—;

wherein X₂ is —O—, —N—, —NR¹⁸—, —CR¹⁹—, or —CO—,

wherein R¹⁸ and R¹⁹ are hydrogen, alkyl, alkenyl, alkynyl; or X₂ together with R¹ and the atom to which R¹ is attached form a heterocyclic ring having 4 to 8 ring members with at least one heteroatom selected from oxygen or nitrogen;

wherein X₄ is —O—, —CR²⁰—, —CO—, or —N—, wherein R²⁰ is hydrogen, alkyl, alkenyl, alkynyl, or hydroxy; or wherein X₄ together with R¹ and the atoms to which they are attached form a heterocyclic ring having 4 to 8 ring members with at least one heteroatom selected from oxygen or nitrogen;

wherein X₅, is —CR²¹— or —N—, wherein R²¹ is hydrogen, alkyl, alkenyl, alkynyl;

wherein X₆, is —CR²²— or —N—, wherein R22 is hydrogen, alkyl, alkenyl, alkynyl, alkoxy, aryl, aralkyl, halogen, or nitro; or X₆ together with X₉ and the carbon at position 7 form a heterocylic ring having 4 to 8 ring members with at least one heteroatom selected from oxygen or nitrogen;

wherein X₈, is —CR²³— or —N—, wherein R²³ is hydrogen, alkyl, alkenyl, alkynyl;

wherein X₉ is alkyl, alkenyl, alkynyl, ether, secondary or tertiary amino, or sulfanyl; or X₉ together with X₆ and the carbon at position 7 form a heterocylic ring having 4 to 8 ring members with at least one heteroatom selected from oxygen or nitrogen;

and wherein at least one of X₁, X₂, X₄, X₅, X₆, X₈ is not —CR—; and wherein n is 0, 1, 2, or 3.

Molecular terms, when used in this application, have their common meaning unless otherwise specified. It should be noted that the alphabetical letters used in the formulas of the present invention should be interpreted as the functional groups, moieties, or substituents as defined herein. Unless otherwise defined, the symbols will have their ordinary and customary meaning to those skilled in the art.

The term “acyl” refers to —COR wherein R used in this definition is hydrogen, alkyl, alkenyl, alkynyl, carbocyclic, heterocylic, aryl, or aralkyl. Most preferably, R is hydrogen, alkyl, aryl, or aralkyl.

The term “amido” indicates either a C-amido group such as —CONR′R″ or an N-amido group such as —NR′COR″ wherein R′ and R″ as used in this definition are independently hydrogen, alkyl, alkenyl, alkynyl, alkoxy, carbocyclic, heterocylic, aryl, or aralkyl. A “sulfonamido” group includes the —NR′—SO₂—R″. Most preferably, R′ and R″ are hydrogen, alkyl, aryl, or aralkyl.

The term “amino” refers to a primary, secondary or tertiary amino group of the formula —NR′R″ wherein R′ and R″ as used in this definition are independently hydrogen, alkyl, alkyenyl, alkynyl, aralkyl, carbocyclic, heterocyclic, aralkyl, or other amino (in the case of hydrazide) or R′ and R″ together with the nitrogen atom to which they are attached, form a ring having 4 to 8 atoms. Thus, the term “amino,” as used herein, includes unsubstituted, monosubstituted (e.g., monoalkylamino or monoarylamino), and disubstituted (e.g., dialkylamino or aralkylamino) amino groups. Amino groups include —NH₂, methylamino, ethylamino, dimethylamino, diethylamino, methyl-ethylamino, pyrrolidin-1-yl, or piperidino, morpholino, etc. Other exemplary “amino” groups forming a ring include pyrrolyl, imidazolyl, pyrazolyl, isothiazolyl, isoxazolyl, pyridyl, pyrazinyl, pyrimidinyl, pyridazinyl, indolizinyl, isoindolyl, indolyl, indazolyl, purinyl, quinolizinyl. The ring containing the amino group may be optionally substituted with another amino, alkyl, alkenyl, alkynyl, halo, or hydroxy group.

The term “alkyl” refers to a branched or unbranched saturated hydrocarbon group of 1 to 24 carbon atoms, such as methyl, ethyl, n-propyl, isopropyl, n-butyl, isobutyl, t-butyl, octyl, decyl, tetradecyl, hexadecyl, eicosyl, tetracosyl, and the like. Preferred “alkyl” groups herein contain 1 to 12 carbon atoms. Most preferred are “lower alkyl” which refer to an alkyl group of one to six, more preferably one to four, carbon atoms. The alkyl group may be optionally substituted with an amino, alkyl, halo, or hydroxy group.

The term “alkoxy” denotes oxy-containing groups substituted with an alkyl, or cycloalkyl group. Examples include, without limitation, methoxy, ethoxy, tert-butoxy, and cyclohexyloxy. Most preferred are “lower alkoxy” groups having one to six carbon atoms. Examples of such groups include methoxy, ethoxy, propoxy, butoxy, isopropoxy, and tert-butoxy groups.

The terms “alkenyl” and “alkynyl” refer to unsaturated aliphatic groups analogous in length and possible substitution to the alkyls described above, but that contain at least one double bond or triple bond respectively.

The term “aryl” means a carbocyclic aromatic system containing one, two, or three rings wherein such rings may be attached together in a pendant manner or may be fused. The term “fused” means that a second ring is present (i.e., attached or formed) by having two adjacent atoms in common (i.e., shared) with the first ring. The term “fused” is equivalent to the term “condensed.” The term “aryl” embraces aromatic groups such as phenyl, naphthyl, tetrahydronaphthyl, indane, and biphenyl. The aryl group may optionally be substituted with an amino, alkyl, halo, hydroxy, carbocyclic, heterocyclic, or another aryl group.

The term “aralkyl” embraces aryl-substituted alkyl moieties. Preferable aralkyl groups are “lower aralkyl” groups having aryl groups attached to alkyl groups having one to six carbon atoms. Examples of such groups include benzyl, diphenylmethyl, triphenylmethyl, phenylethyl, and diphenylethyl. The terms benzyl and phenylmethyl are interchangeable.

The term “aryloxy” embraces aryl groups, as defined above, attached to an oxygen atom. The aryloxy groups may optionally be substituted with a halo, hydroxy, or alkyl group. Examples of such groups include phenoxy, 4-chloro-3-ethylphenoxy, 4-chloro-3-methylphenoxy, 3-chloro-4-ethylphenoxy, 3,4-dichlorophenoxy, 4-methylphenoxy, 3-trifluoromethoxyphenoxy, 3-trifluoromethylphenoxy, 4-fluorophenoxy, 3,4-dimethylphenoxy, 5-bromo-2-fluorophenoxy, 4-bromo-3-fluorophenoxy, 4-fluoro-3-methylphenoxy, 5,6,7,8-tetrahydronaphthyloxy, 3-isopropylphenoxy, 3-cyclopropylphenoxy, 3-ethylphenoxy, 4-tert-butylphenoxy, 3-pentafluoroethylphenoxy, and 3-(1,1,2,2-tetrafluoroethoxy)phenoxy.

The term “aralkoxy” embraces oxy-containing aralkyl groups attached through an oxygen atom to other groups. “Lower aralkoxy” groups are those phenyl groups attached to lower alkoxy group as described above. Examples of such groups include benzyloxy, 1-phenylethoxy, 3-trifluoromethoxybenzyloxy, 3-trifluoromethylbenzyloxy, 3,5-difluorobenyloxy, 3-bromobenzyloxy, 4-propylbenzyloxy, 2-fluoro-3-trifluoromethylbenzyloxy, and 2-phenylethoxy.

The term “carboxyl” refers to —R′C(═O)OR″, wherein R′ and R″ as used in this definition are independently hydrogen, alkyl, alkenyl, alkynyl, carbocyclic, heterocylic, aryl, or aralkyl or R′ can additionally be a covalent bond. “Carboxyl” includes both carboxylic acids, and carboxylic acid esters. The term “carboxylic acid” refers to a carboxyl group in which R″ is hydrogen. Such acids include formic, acetic, propionic, butyric, valeric acid, 2-methyl propionic acid, oxirane-carboxylic acid, and cyclopropane carboxylic acid. The term “carboxylic acid ester” or “ester” refers to a carboxyl group in which R″ is alkyl, alkenyl, alkynyl, carbocyclic, heterocylic, aryl, or aralkyl.

The term “carbocyclic” refers to a group that contains one or more covalently closed ring structures, and that the atoms forming the backbone of the ring are all carbon atoms. The ring structure may be saturated or unsaturated. The term thus distinguishes carbocyclic from heterocyclic rings in which the ring backbone contains at least one non-carbon atom. The term carbocylic encompasses cycloalkyl ring systems.

The terms “cycloalkane” or “cyclic alkane” or “cycloalkyl” refer to a carbocyclic group in which the ring is a cyclic aliphatic hydrocarbon, for example, a cyclic alkyl group preferably with 3 to 12 ring carbons. “Cycloalkyl” includes, by way of example, cyclopropyl, cyclobutyl, cyclopentyl, cyclohexyl, cycloheptyl, or cyclooctyl, and the like. The cycloalkyl group may be optionally substituted with an amino, alkyl, halo, or hydroxy group.

The term “ether” refers to the group —R′—O—R″ wherein R′ and R″ as used in this definition are independently hydrogen, alkyl, alkenyl, alkynyl, carbocyclic, heterocylic, aryl, or aralkyl, and R′ can additionally be a covalent bond attached to a carbon.

The terms “halo” or “halogen” refer to fluoro, chloro, bromo, or iodo, usually regarding halo substitution for a hydrogen atom in an organic compound.

The term “heterocyclic or heterocycle” means an optionally substituted, saturated or unsaturated, aromatic or non-aromatic cyclic hydrocarbon group with 4 to about 12 carbon atoms, preferably about 5 to about 6, wherein 1 to about 4 carbon atoms are replaced by nitrogen, oxygen or sulfur. Exemplary heterocyclic which are aromatic include groups pyridinyl, furanyl, benzofuranyl, isobenzofuranyl, pyrrolyl, thienyl, 1,2,3-triazolyl, 1,2,4-triazolyl, indolyl, imidazolyl, thiazolyl, thiadiazolyl, pyrimidinyl, oxazolyl, triazinyl, and tetrazolyl. Exemplary heterocycles include benzimidazole, dihydrothiophene, dioxin, dioxane, dioxolane, dithiane, dithiazine, dithiazole, dithiolane, furan, indole, 3-H indazole, 3-H-indole, imidazole, indolizine, isoindole, isothiazole, isoxazole, morpholine, oxazole, oxadiazole, oxathiazole, oxathiazolidine, oxazine, oxadiazine, piperazine, piperidine, purine, pyran, pyrazine, pyrazole, pyridine, pyrimidine, pyrimidine, pyridazine, pyrrole, pyrrolidine, tetrahydrofuran, tetrazine, thiadiazine, thiadiazole, thiatriazole, thiazine, thiazole, thiomorpholine, thiophene, thiopyran, triazine, and triazole. The heterocycle may be optionally substituted with an amino, alkyl, alkenyl, alkynyl, halo, hydroxy, carbocyclic, thio, other heterocyclic, or aryl group. Exemplary heterocyclic groups include 1-pyrrolyl, 2-pyrrolyl, 3-pyrrolyl, 1-indolyl, 2-indolyl, 3-indolyl, 1-pyridyl, 2-pyridyl, 3-pyridyl, 4-pyridyl, 1-imidazolyl, 2-imidazolyl, 3-imidazolyl, 4-imidazolyl, 1-pyrazolyl, 2 pyrazolyl, 3-pyrazolyl, 4-pyrazolyl, 5-pyrazolyl, 1-pyrazinyl, 2-pyrazinyl, 1-pyrimidinyl, 2-pyrimidinyl, 4-pyrimidinyl, 5-pyrimidinyl, 1-pyridazinyl, 2-pyridazinyl, 3-pyridazinyl, 4-pyridizinyl, 1-indolizinyl, 2-indolizinyl, 3-indolizinyl, 4-indolizinyl, 5-indolizinyl, 6-indolizinyl, 7-indolizinyl, 8-indolizinyl, 1-isoindolyl, 2-isoindolyl, 3-isoindolyl, 4-isoindolyl, 5-isoindolyl.

The term “hydroxy” or “hydroxyl” refers to the substituent —OH.

The term “oxo” shall refer to the substituent ═O.

The term “nitro” means —NO₂

The term “sulfanyl” refers to —SR′ where R′ as used in this definition is hydrogen, alkyl, alkenyl, alkynyl, carbocyclic, heterocylic, aryl, or aralkyl.

The term “sulfenyl” refers to —SOR′ where R′ as used is this definition is hydrogen, alkyl, alkenyl, alkynyl, carbocyclic, heterocylic, aryl, or aralkyl.

The term “sulfonyl” refers to —SOR′ where R′ as used in this definition is hydrogen, alkyl, alkenyl, alkynyl, carbocyclic, heterocylic, aryl, or aralkyl.

“Optional” or “optionally” means that the subsequently described event or circumstance may or may not occur, and that the description includes instances where said event or circumstance occurs and instances in which it does not. “Optionally” is inclusive of embodiments in which the described conditions is present and embodiments in which the described condition is not present. For example, “optionally substituted phenyl” means that the phenyl may or may not be substituted, and that the description includes both unsubstituted phenyl and phenyl wherein there is substitution. “Optionally” is inclusive of embodiments in which the described conditions is present and embodiments in which the described condition is not present.

The compounds of the present invention can exist in tautomeric, geometric, or stereoisomeric forms. The present invention contemplates all such compounds, including cis- and trans-geometric isomers, E- and Z-geometric isomers, R- and S-enantiomers, diastereomers, d-isomers, 1-isomers, the racemic mixtures thereof and other mixtures thereof, as falling within the scope of the invention.

In one aspect, the present disclosure is directed to methods utilizing compounds of Formula (I) wherein X₁ is —O— and X₂ is —CO—.

In another aspect, the present disclosure is directed to methods utilizing compounds of Formula (I) wherein X₄, X₅, and X₆ are CH, and X₈ is CH or C—CH₃.

In a further aspect, the present disclosure is directed to methods utilizing compounds of Formula (I) wherein n=1, R₂ and R₃ are —OH , R₄ and R₅ are CH₃, and wherein R₆ is —O— CH₃.

In another aspect, the present disclosure is directed to methods utilizing compounds of Formula (I) wherein R¹ is amido which is —NHCOCH₃.

In a further aspect, the present disclosure is directed to methods utilizing compounds of Formula (I) wherein X₉ is —O—.

In one embodiment, the methods of the disclosure utilize an Hsp90 inhibitor which is selected from: N-(7-((3aR,4R,7R,7aR)-7-methoxy-6,6-dimethyl-2-oxo-tetrahydro-3aH-[1,3]dioxolo[4,5-c]pyran-4-yloxy)-2-oxo-2H-chromen-3-yl)acetamide (A1); (2R,3R,4R, 5R)-2-(3-acetamido-2-oxo-2H-chromen-7-yloxy)-4-hydroxy-5-methox-y-6,6-dimethyl-tetrahydro-2H-pyran-3-yl carbamate (A2); (3R,4S,5R,6R)-6-(3-acetamido-2-oxo-2H-chromen-7-yl hydroxy-3-methoxy-2,2-dimethyl tetrahydro-2H-pyran-4-yl carbamate (A3); N-(7-((2R,3R,4S,5R)-3,4-dihydroxy-5-methoxy-6,6-dimethyl-tetrahydro-2H-pyran-2-yloxy)-2-oxo-2H-chromen-3-yl)acetamide (A4); 7-((3aR,4R,7R,7aR)-7-methoxy-6,6-dimethyl-2-oxo-tetrahydro-3aH-[1,3]dioxolo[4,5-c]pyran-4-yloxy)-2H-chromen-2-one (B1); (3R,4S,5R,6R)-5-hydroxy-3-methoxy-2,2-dimethyl-6-(2-oxo-2H-chromen-7-yloxy)-tetrahydro-2H-pyran-4-yl carbamate (B2); (2R,3R,4R,5R)-4-hydroxy-5-methoxy-6,6-dimethyl-2-(2-oxo-2H-chromen-7-yloxy)-tetrahydro-2H-pyran-3-yl carbamate (B3); 7-((2R,3R,4S,5R)-3,4-dihydroxy-5-methoxy-6,6-dimethyl-tetrahydro-2H-pyran-2-yloxy)-2H-chromen-2-one (B4); 7-((3aR,4R,7R,7aR)-7-methoxy-6,6-dimethyl-2-oxo-tetrahydro-3aH-[1,3]dioxolo[4,5-c]pyran-4-yloxy)-4-methyl-3-phenyl-2H-chromen-2-one (C1); (3R,4S,5R,6R)-5-hydroxy-3-methoxy-2,2-dimethyl-6-(6-(4-methyl-2-oxo-3-phenyl-2H-chromen-7-yloxy)-tetrahydro-2H-pyran-4-yl carbamate (C2); (2R,3R,4R,5R)-4-hydroxy-5-methoxy-6,6-dimethyl-2-(4-methyl-2-oxo-3-phenyl-2H-chromen-7-yloxy)-tetrahydro-2H-pyran-3-yl carbamate (C3); 7-((2R,3R,4S,5R)-3,4-dihydroxy-5-methoxy-6,6-dimethyl-tetrahydro-2H-pyran-2-yloxy)-4-methyl-3-phenyl-2H-chromen-2-one (C4); 8-(7-Methoxy-6,6-dimethyl-2-oxo-tetrahydro-[1,3]dioxolo[4,5-c]pyran-4-yloxy)-chromen-2-one (D1); Carbamic acid 4-hydroxy-5-methoxy-6,6-dimethyl-2-(2-oxo-2H-chromen-8-yloxy)-tetrahydro-pyran-3-yl ester (D2); carbamic acid 5-hydroxy-3-methoxy-2,2-dimethyl-6-(2-oxo-2H-chromen-8-yloxy)-tetrahydro-pyran-4-yl ester (D3); 8-(3,4-Dihydroxy-5-methoxy-6,6-dimethyl-tetrahydro-pyran-2-yloxy)-chromen-2-one (D4); 6-(7-Methoxy-6,6-dimethyl-2-oxo-tetrahydro-[1,3]dioxolo[4,5-c]pyran-4-yloxy)-chromen-2-one (E1); Carbamic acid 5-hydroxy-3-methoxy-2,2-dimethyl-6-(2-oxo-2H-chromen-6-yloxy)-tetrahydro-pyran-4-yl ester (E2); Carbamic acid 4-hydroxy-5-methoxy-6,6-dimethyl-2-(2-oxo-2H-chromen-6-yloxy)-tetrahydro-pyran-3-yl ester (E3); and 6-(3,4-Dihydroxy-5-methoxy-6,6-dimethyl-tetrahydro-pyran-2-yloxy)-chromen-2-one (E4). In specific embodiments, the Hsp90 inhibitor is N-(7-((2R,3R,4S,5R)-3,4-dihydroxy-5-methoxy-6,6-dimethyl-tetrahydro-2H-py-ran-2-yloxy)-2-oxo-2H-chromen-3-yl)acetamide (A4). In other specific embodiments, the Hsp90 inhibitor is N-(7-((2R,3R,4S,5R)-3,4-dihydroxy-5-methoxy-6,6-dimethyltetrahydro-2H-pyran-2-yloxy)-8-methyl-2-oxo-2H-chromen-3-yl)acetamide (KU-32). KU-32 can be prepared, for example, in an analogous fashion to the preparation of compound A4 of Blagg et al., U.S. Patent Application Publication 2007/0270452, Example 1. For example, 7-hydroxy-8-methyl-3-acetamidocoumarin can be coupled with a trichloroacetimidate of noviose carbonate in the presence of boron trifluoride etherate; the resulting cyclic carbonate can be treated with methanolic ammonia to provide the crude desired compound, which can be purified by, for example, silica gel chromatography and/or HPLC. The coumarin intermediate, 7-hydroxy-8-methyl-3-acetamidocoumarin, can be prepared, for example, by condensation of commercially available 2,4-dihydroxy-3-methylbenzaldehyde with glycine in the presence of acetic anhydride, followed by cleavage of the O-acetyl group with potassium carbonate in, for example, methanol.

In one embodiment, the present disclosure is directed to methods utilizing compounds of Formula (I) wherein R¹ is an amido which is NR′COR″, and R′ is hydrogen and R″ is aryl according to:

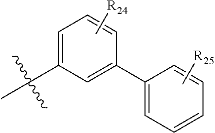

wherein R²⁴ and R²⁵ are independently hydrogen, alkyl, amino, halo, hydroxy, or alkoxy.

In another embodiment, the methods of the disclosure utilize an Hsp90 inhibitor which is selected from: N-(7-((2R,3R,4S,5R)-3,4-dihydroxy-5-methoxy-6,6-dimethyltetrahydro-2H-pyran-2-yloxy)-8-methyl-2-oxo-2H-chromen-3yl)-6-methoxybiphenyl-3-carboxamide (28);

N-(7-((2R,3R,4S,5R)-3,4-dihydroxy-5-methoxy-6,6-dimethyltetrahydro-2H-pyran-2-yloxy)-8-methyl-2-oxo-2H-chromen-3-yl)-6-methoxy-2′-methylbiphenyl-3-carboxamide (29);

N-(7-((2R,3R,4S,5R)-3,4-dihydroxy-5-methoxy-6,6-dimethyltetrahydro-2H-pyran-2-yloxy)-8-methyl-2-oxo-2H-chromen-3-yl)-6-methoxy-3′-methylbiphenyl-3-carboxamide (30);

N-(7-((2R,3R,4S,5R)-3,4-dihydroxy-5-methoxy-6,6-dimethyltetrahydro-2H-pyran-2-yloxy)-8-methyl-2-oxo-2H-chromen-3-yl)-6-methoxy-4′-methylbiphenyl-3-carboxamide (31);

N-(7-((2R,3R,4S,5R)-3,4-dihydroxy-5-methoxy-6,6-dimethyltetrahydro-2H-pyran-2-yloxy)-8-methyl-2-oxo-2H-chromen-3-yl)-2′,6-dimethoxybiphenyl-3-carboxamide (32);

N-(7-((2R,3R,4S,5R)-3,4-dihydroxy-5-methoxy-6,6-dimethyltetrahydro-2H-pyran-2-yloxy)-8-methyl-2-oxo-2H-chromen-3-yl)-3′,6-dimethoxybiphenyl-3-carboxamide (33);

N-(7-((2R,3R,4S,5R)-3,4-dihydroxy-5-methoxy-6,6-dimethyltetrahydro-2H-pyran-2-yloxy)-8-methyl-2-oxo-2H-chromen-3-yl)-4′,6-dimethoxybiphenyl-3-carboxamide (34);

N-(7-((2R,3R,4S,5R)-3,4-dihydroxy-5-methoxy-6,6-dimethyltetrahydro-2H-pyran-2-yloxy)-8-methyl-2-oxo-2H-chromen-3-yl)-2′-hydroxy-6-methoxybiphenyl-3-carboxamide (35);

N-(7-((2R,3R,4S,5R)-3,4-dihydroxy-5-methoxy-6,6-dimethyltetrahydro-2H-pyran-2-yloxy)-8-methyl-2-oxo-2H-chromen-3-yl)-3′-hydroxy-6-methoxybiphenyl-3-carboxamide (36);

N-(7-((2R,3R,4S,5R)-3,4-dihydroxy-5-methoxy-6,6-dimethyltetrahydro-2H-pyran-2-yloxy)-8-methyl-2-oxo-2H-chromen-3-yl)-4′-hydroxy-6-methoxybiphenyl-3-carboxamide (37)

N-(7-((2S,3R,4S,5R)-3,4-dihydroxy-5-methoxy-6,6-dimethyltetrahydro-2H-pyran-2-yloxy)-6-methoxy-8-methyl-2-oxo-2H-chromen-3-yl)-3′,6-dimethoxybiphenyl-3-carboxamide (26a);

N-(7-((2S,3R,4S,5R)-3,4-dihydroxy-5-methoxy-6,6-dimethyltetrahydro-2H-pyran-2-yloxy)-8-methyl-2-oxo-6-propoxy-2H-chromen-3-yl)-3′,6-dimethoxybiphenyl-3-carboxamide (26b);

N-(7-((2S,3R,4S,5R)-3,4-dihydroxy-5-methoxy-6,6-dimethyltetrahydro-2H-pyran-2-yloxy)-6-isopropoxy-8-methyl-2-oxo-2H-chromen-3-yl)-3′,6-dimethoxybiphenyl-3-carboxamide (26c);

N-(7-((2R,3R,4S,5R)-3,4-dihydroxy-5-methoxy-6,6-dimethyltetrahydro-2H-pyran-2-yloxy)-5-methoxy-8-methyl-2-oxo-2H-chromen-3-yl)-3′,6-dimethoxybiphenyl-3-carboxamide (26d);

N-(8-benzyl-7-((2R,3R,4S,5R)-3,4-dihydroxy-5-methoxy-6,6-dimethyltetrahydro-2H-pyran-2-yloxy)-2-oxo-2H-chromen-3-yl)-3′,6-dimethoxybiphenyl-3-carboxamide (26e);

N-(7-((2R,3R,4S,5R)-3,4-dihydroxy-5-methoxy-6,6-dimethyltetrahydro-2H-pyran-2-yloxy)-2-oxo-8-phenyl-2H-chromen-3-yl)-3′,6-dimethoxybiphenyl-3-carboxamide (26f);

N-(7-((2S,3R,4S,5R)-3,4-dihydroxy-5-methoxy-6,6-dimethyltetrahydro-2H-pyran-2-yloxy)-8-methoxy-2-oxo-2H-chromen-3-yl)-3′,6-dimethoxybiphenyl-3-carboxamide (26g);

N-(7-((2R,3R,4S,5R)-3,4-dihydroxy-5-methoxy-6,6-dimethyltetrahydro-2H-pyran-2-yloxy)-8-ethyl-2-oxo-2H-chromen-3-yl)-3′,6-dimethoxybiphenyl-3-carboxamide (26h);

N-(7-((2S,3R,4S,5R)-3,4-dihydroxy-5-methoxy-6,6-dimethyltetrahydro-2H-pyran-2-yloxy)-6-methoxy-8-methyl-2-oxo-2H-chromen-3-yl)-1H-indole-2-carboxamide (26i);

N-(7-((2S,3R,4S,5R)-3,4-dihydroxy-5-methoxy-6,6-dimethyltetrahydro-2H-pyran-2-yloxy)-8-methyl-2-oxo-6-propoxy-2H-chromen-3-yl)-1H-indole-2-carboxamide (26j);

N-(7-((2S,3R,4S,5R)-3,4-dihydroxy-5-methoxy-6,6-dimethyltetrahydro-2H-pyran-2-yloxy)-6-isopropoxy-8-methyl-2-oxo-2H-chromen-3-yl)-1H-indole-2-carboxamide (26k);

N-(7-((2R,3R,4S,5R)-3,4-dihydroxy-5-methoxy-6,6-dimethyltetrahydro-2H-pyran-2-yloxy)-5-methoxy-8-methyl-2-oxo-2H-chromen-3-yl)-1H-indole-2-carboxamide (26l);

N-(8-benzyl-7-((2R,3R,4S,5R)-3,4-dihydroxy-5-methoxy-6,6-dimethyltetrahydro-2H-pyran-2-yloxy)-2-oxo-2H-chromen-3-yl)-]H-indole-2-carboxamide (26m);

N-(7-((2R,3R,4S,5R)-3,4-dihydroxy-5-methoxy-6,6-dimethyltetrahydro-2H-pyran-2-yloxy)-2-oxo-8-phenyl-2H-chromen-3-yl)-1H-indole-2-carboxamide (26n);

N-(7-((2S,3R,4S,5R)-3,4-dihydroxy-5-methoxy-6,6-dimethyltetrahydro-2H-pyran-2-yloxy)-8-methoxy-2-oxo-2H-chromen-3-yl)-1H-indole-2-carboxamide (26o);

N-(7-((2R,3R,4S,5R)-3,4-dihydroxy-5-methoxy-6,6-dimethyltetrahydro-2H-pyran-2-yloxy)-8-ethyl-2-oxo-2H-chromen-3-yl)-1H-indole-2-carboxamide (26p) N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)-2-phenylacetamide (22);

N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)-3-phenylpropanamide (23);

Benzyl 7-((2R,3R,4S,5R)-3,4-dihydroxy-5-methoxy-6,6-dimethyltetrahydro-2H-pyran-2-yloxy)-8-methyl-2-oxo-2H-chromen-3-ylcarbamate (24);

N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)cinnamamide (25);

N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)picolinamide (40);

N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)nicotinamide (41);

N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)isonicotinamide (42);

N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)benzofuran-2-carboxamide (43);

N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)-1H-indole-2-carboxamide (46);

N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)-1H-indole-3-carboxamide (47);

N-(7-((2R,3R,4S,5R)-3,4-Dihydroxy-5-methoxy-6,6-dimethyl-tetrahydro-2H-pyran-2-yloxy)quinolin-3-yl)-4-methoxy-3-(3-methoxyphenyl)-benzamide (42b);

N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)benzamide (8);

N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)biphenyl-2-carboxamide (12);

N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)biphenyl-3-carboxamide (13);

2-Amino-N-(7-((2R,3R,4S,5R)-3,4-dihydroxy-5-methoxy-6,6-dimethyltetrahydro-2H-pyran-2-yloxy)-8-methyl-2-oxo-2H-chromen-3-yl)benzamide (18);

3-Amino-N-(7-((2R,3R,4S,5R)-3,4-dihydroxy-5-methoxy-6,6-dimethyltetrahydro-2H-pyran-2-yloxy)-8-methyl-2-oxo-2H-chromen-3-yl)benzamide (19);

4-Amino-N-(7-((2R,3R,4S,5R)-3,4-dihydroxy-5-methoxy-6,6-dimethyltetrahydro-2H-pyran-2-yloxy)-8-methyl-2-oxo-2H-chromen-3-yl)benzamide (20);

N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)-2-methoxybenzamide (9);

N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)-3-methoxybenzamide (10); and

N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)-4-methoxybenzamide (11).

In one specific aspect, the Hsp90 inhibitor is selected from N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)-1H-indole-2-carboxamide (46); and N-(7-((2R,3R,4S,5R)-3,4-Dihydroxy-5-methoxy-6,6-dimethyl-tetrahydro-2H-pyran-2-yloxy)quinolin-3-yl)-4-methoxy-3-(3-methoxyphenyl)-benzamide (42b).

In another embodiment, the Hsp90 inhibitor is selected from the group consisting of 3-(3′,6-dimethoxybiphenyl-3-ylcarboxamido)-8-methyl-2-oxo-2H-chromen-7-yl propionate; 3-(3′,6-dimethoxybiphenyl-3-ylcarboxamido)-8-methyl-2-oxo-2H-chromen-7-yl cyclopropane carboxylate; and 3-(3′,6-dimethoxybiphenyl-3-ylcarboxamido)-6-methoxy-8-methyl-2-oxo-2H-chromen-7-yl acetate, respectively shown below:

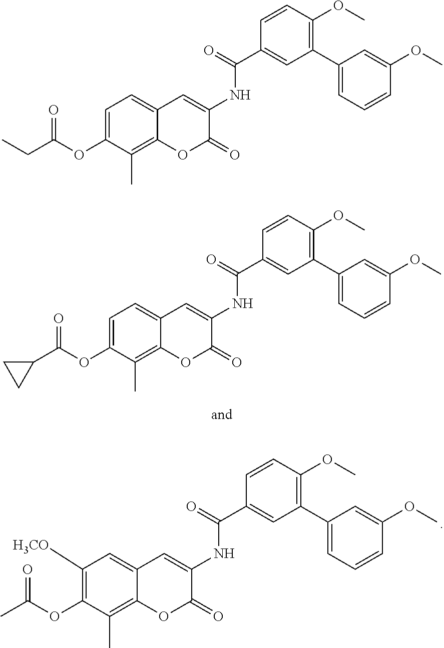

As discussed therein, the compounds to be used in the dosing methods of the present invention can exist in tautomeric, geometric, or stereoisomeric forms. The present invention contemplates all such compounds, including cis- and trans-geometric isomers, E- and Z-geometric isomers, R- and S-enantiomers, diastereomers, d-isomers, 1-isomers, the racemic mixtures thereof and other mixtures thereof, as falling within the scope of the invention.

Also included in the family of compounds of the present invention are the pharmaceutically acceptable salts, esters, and prodrugs thereof. The term “pharmaceutically-acceptable salts” embraces salts commonly used to form alkali metal salts and to form addition salts of free acids or free bases. The nature of the salt is not critical, provided that it is pharmaceutically acceptable. Suitable pharmaceutically acceptable acid addition salts of compounds of the present invention be prepared from inorganic acid or from an organic acid. Examples of such inorganic acids are hydrochloric, hydrobromic, hydroiodic, nitric, carbonic, sulfuric, and phosphoric acid. Appropriate organic acids may be selected from aliphatic, cycloaliphatic, aromatic, araliphatic, heterocyclic, carboxylic and sulfonic classes of organic acids, examples of which are formic, acetic, propionic, succinic, glycolic, gluconic, lactic, malic, tartaric, citric, ascorbic, glucoronic, maleic, fumaric, pyruvic, aspartic, glutamic, benzoic, anthranilic, mesylic, salicylic, p-hydroxybenzoic, phenylacetic, mandelic, embonic (pamoic), methanesulfonic, ethylsulfonic, benzenesulfonic, sulfanilic, stearic, cyclohexylaminosulfonic, algenic, and galacturonic acid. Suitable pharmaceutically-acceptable base addition salts of compounds of the present invention include metallic salts made from aluminum, calcium, lithium, magnesium, potassium, sodium and zinc or organic salts made from N,N′-dibenzylethyleneldiamine, choline, chloroprocaine, diethanolamine, ethylenediamine, meglumine (N-methylglucamine) and procain. All of these salts may be prepared by conventional means from the corresponding compounds of by reacting, for example, the appropriate acid or base with the compounds of the present invention.

As used herein, the term “pharmaceutically acceptable ester” refers to esters which hydrolyze in vivo and include, but are not limited to, those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include formates, acetates, propionates, butyrates, acrylates, and ethylsuccinates.

The term “pharmaceutically acceptable prodrugs” as used herein refers to those prodrugs of the compounds of the present invention which are, within the scope of sound medical judgment, suitable for use in contact with the tissues of humans and lower animals without undue toxicity, irritation, allergic response and the like, commensurate with a reasonable risk/benefit ratio, and effective for their intended use, where possible, of the compounds of the invention. The term “prodrug” refers to compounds that are rapidly transformed in vivo to yield the parent compound of the above formulae, for example, by hydrolysis in blood. A thorough discussion is provided in T. Higuchi and V. Stella, Prodrugs as Novel delivery Systems, Vol. 14 of the A.C.S. Symposium Series and in Edward B. Roche, ed., Bioreversible Carriers in Drug Design, American Pharmaceutical Association and Pergamon Press, (1987), both of which are incorporated by reference herein.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutical Compositions Used In the Dosing Methods

As described above, a “dose” according to the disclosure comprises a therapeutically effective amount of at least one Hsp90 inhibitor. Non-limiting examples of Hsp90 inhibitors which may be utilized to provide a dose are described in detail above in the section entitled “Hsp90 Inhibitor Compounds.” Preferred Hsp90 inhibitors include N-(7-((2R,3R,4S,5R)-3,4-dihydroxy-5-methoxy-6,6-dimethyl-tetrahydro-2H-py-ran-2-yloxy)-2-oxo-2H-chromen-3-yl)acetamide (A4, KU-1) and N-(7-((2R,3R,4S,5R)-3,4-dihydroxy-5-methoxy-6,6-dimethyltetrahydro-2H-pyran-2-yloxy)-8-methyl-2-oxo-2H-chromen-3-yl)acetamide (KU-32).

Preferably, a dose is administered in the form of a pharmaceutical composition which comprises at least one Hsp90 inhibitor (for example, KU-1 and/or KU-32) and also optionally includes one or more pharmaceutically acceptable carriers. The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject novobiocin analogue or derivative from one organ, or portion of the body, to another organ, or portion of the body. Each carrier must be “acceptable” in the sense of being compatible with the other ingredients of the formulation and not injurious to the patient. Some examples of materials which may serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. Preferred carriers are cyclodextrins, such as sulfobutyl ether β-cyclodextrins (including the Captisol® brand hepta-substituted sulfobutyl ether β-cyclodextrin available from Cydex Pharmaceuticals, Inc.), and those described in U.S. Pat. Nos. 6,046,177; 5,874,418; 5,376,645; and 5,134,127, which are incorporated by reference.

The pharmaceutical composition may comprise other active pharmaceutical ingredients in addition to the one or more Hsp90 inhibitors. In addition, the pharmaceutical compositions may be administered in conjunction with other active pharmaceutical ingredients in separate dosage forms. The other active pharmaceutical ingredients may be administered on a more traditional dosing regimen, such as daily or twice daily.

The compositions may be formulated for any route of administration, in particular for oral, rectal, transdermal, subcutaneous, intravenous, intramuscular, intranasal, or intraperitoneal administration. The compositions may be formulated in any conventional form, for example, as tablets, capsules, caplets, solutions, suspensions, dispersions, syrups, sprays, gels, suppositories, patches, and emulsions.

As described above, a dose comprises a therapeutically effective amount of the Hsp90 inhibitor(s). Depending on the quantity of the Hsp90 inhibitor contained in each unit form of a pharmaceutical composition, it may be necessary to administer a plurality of units in order to administer a dose. For example, if the unit form of a pharmaceutical composition is a capsule comprising the Hsp90 inhibitor, a dose may comprise a single capsule or a plurality of capsules, depending on the quantity of Hsp90 inhibitor in each capsule. In addition, a dose may comprise two or more different pharmaceutical compositions administered substantially at the same time (for example, simultaneously or within 2 hours of one another) in order to deliver a therapeutically effective amount of the Hsp90 inhibitor(s). When a single dose comprises two or more different pharmaceutical compositions, those pharmaceutical compositions may be administered by the same route of by a different route. For example, a dose may comprise a capsule for oral administration and a solution for intravenous administration, administered at substantially the same time. Where different pharmaceutical compositions are used for a dose, the pharmaceutical compositions may comprise the same Hsp90 inhibitor(s) in the same or different amounts, or they may comprise different Hsp90 inhibitor(s) in the same or different amounts.

In embodiments where a dosing regimen comprises the administration of more than one dose, it is not necessary that each dose comprises the same pharmaceutical composition or even that each dose is administered by the same route of administration. Broadly, the route of administration and the identity (e.g. the unit form) of the pharmaceutical composition for each of a plurality of doses in a dosing regimen may be selected independently. For example, a first dose may be administered intravenously and subsequent doses may be administered orally. By way of another example, a first dose may be administered orally by one type of pharmaceutical composition (e.g., a capsule) and subsequent doses may be administered orally by another type of pharmaceutical composition (e.g., a suspension). A described above, it is further contemplated that each individual dose may consist of a plurality of different pharmaceutical compositions which deliver a therapeutically effective amount of the Hsp90 inhibitor when administered together.

The following examples are provided to illustrate the present invention and are not intended to limit the scope thereof.

### Examples

### Example 1

### Pharmacokinetic Studies of KU-32

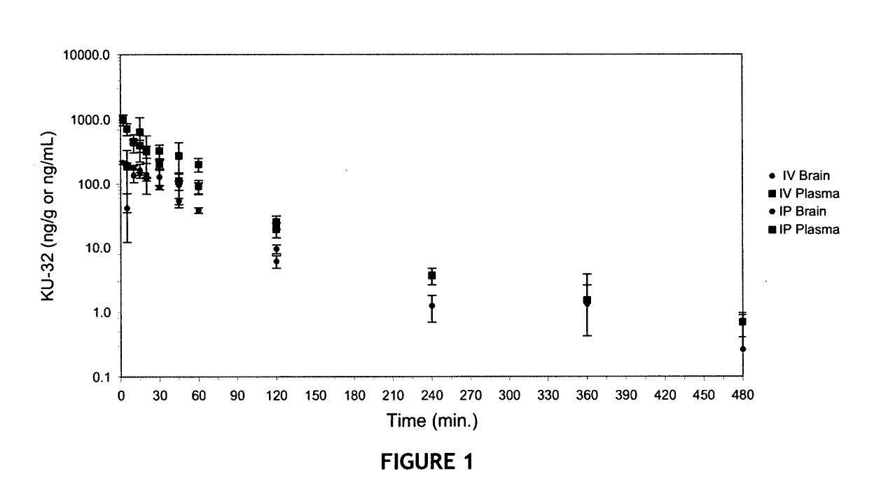

The plasma and brain levels of the Hsp90 inhibitor KU-32 as a function of time in mice were investigated following IP or IV injection to mice. Pharmacokinetics of KU-32 levels in mouse plasma and brain was determined by LC-MS. For these studies, female Balb/c mice (6-7 weeks) were injected with either 2 or 20 mg/kg KU-32 or vehicle. Mice were sacrificed at predetermined time points and plasma was collected following cardiac puncture for determination of KU-32 levels. The animals were perfused with normal saline and the brain was harvested and homogenized. Half the brain was analyzed for KU-32 levels and the remaining half was analyzed for Hsp70 levels. KU-32 levels fall below detectable limits by eight hours as illustrated in FIG. 1.

### Example 2

### KU-32 in vivo Up-Regulation of HSP70

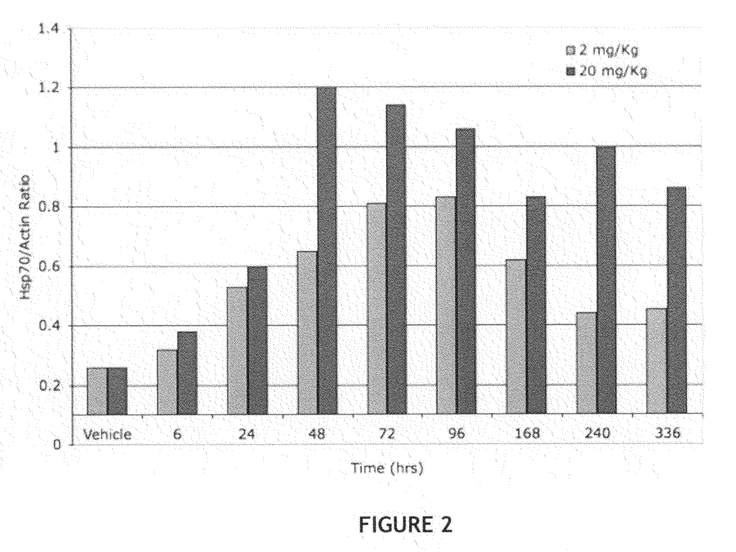

Changes in Hsp70 levels in mouse brain over time following KU-32 IP administration were determined. The ratio of Hsp70 to actin was determined in frontal and hind brain sections recovered from brains of mice at each indicated time point shown in FIG. 2 following administration of either 2 or 20 mg/kg KU-32, as discussed in Example 1. Densitometric analyses were performed, and the ratio of the Hsp70 immunoreactivity was determined using actin as a loading control. Hsp70 levels were increased following single IP injection of KU-32 in mice over a 72-96 hour period and remained elevated, as shown in FIG. 2.

### Example 3

### Daily Dosing Multiple Sclerosis Model

The neurodegenerative disorder multiple sclerosis is often studied in an animal model system termed experimental autoimmune encephalomyelitis (“EAE”). EAE is an inflammatory condition characterized by multifocal perivascular CNS inflammatory infiltrates that primarily include T cells and monocytes. Bar-Or et al., Molecular pathogenesis of multiple sclerosis, Journal of Neuroimmunol. 100:252-259 (1999). EAE can be induced in animals by injection of immunodominant peptides from myelin proteins such as myelin basic protein (MBP), proteolipid protein (PLP), and myelin oligodendrocyte glycoprotein (MOG), or by transfer of CD4+ MHC class II-restricted T-cells reactive with these peptides. Mokhtarian et al., Nature 309: 312-314 (1984); Zamvil et al., T-cell clones specific for myelin basic protein induce chronic relapsing paralysis and demyelination, Nature 317:355-358 (1985). The EAE models are frequently used to study the pathogenesis of MS and to test novel therapeutic strategies aimed at treating MS.

Heat shock response suppresses inflammatory gene expression for nitric oxide synthase, cytokines and chemokines, all of which have been implicated in the development of multiple sclerosis (MS). HSR can be induced by a variety of stresses, including hyperthermia, oxidative stress, heavy metals, viral infection, and UV irradiation. Administration of Hsp90 inhibitors also leads to a HSR due to the dissociation of HSF-1 from Hsp90. Therefore, KU-32, an 8-methyl derivative of KU-1, a novel Hsp90 inhibitor was evaluated in a murine autoimmune disease model (Experimental Autoimmune Encephalomyelitis; EAE) to determine if disease severity and disease incidence is diminished.

The effect of daily dosing of the Hsp90 inhibitor KU-32 following onset of symptoms in a murine MS model was investigated. In this example, female SJL/J mice (6-7 weeks old) were immunized with proteolipid protein (“PLP”) in CFA and pertussis toxin IV on day 0. The mice were boosted with PLP in IFA on day seven.

On day 12 of the study mice received 2, 5 or 10 mg/kg KU-32 by IP injection on a daily basis. KU-32 was formulated at all concentrations in 0.05 M Captisol®. To access the phase-solubility behavior of KU-32 in Captisol®, KU-32 in excess of its expected solubility were equilibrated with increasing concentrations of Captisol® in water. The suspensions were allowed to equilibrate at least 24-hours at 25° C., after which time the suspensions were centrifuged and the KU-32 concentration in the supernatant determined by RP-HPLC. Initial studies exhibited AP-type phase-solubility behavior over the concentration range of 0.05-0.2 M Captisol®. The 0.05 M Captisol® provided adequate solubility for planned studies.

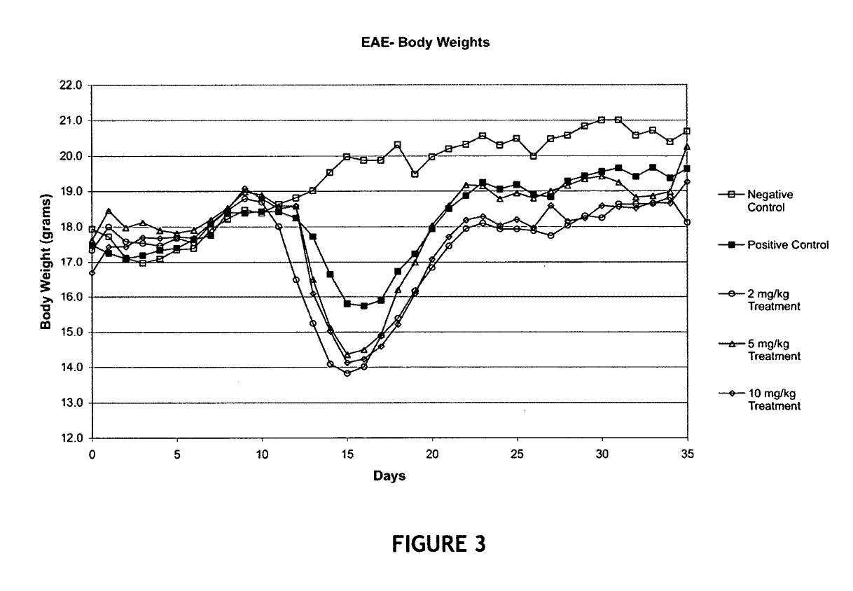

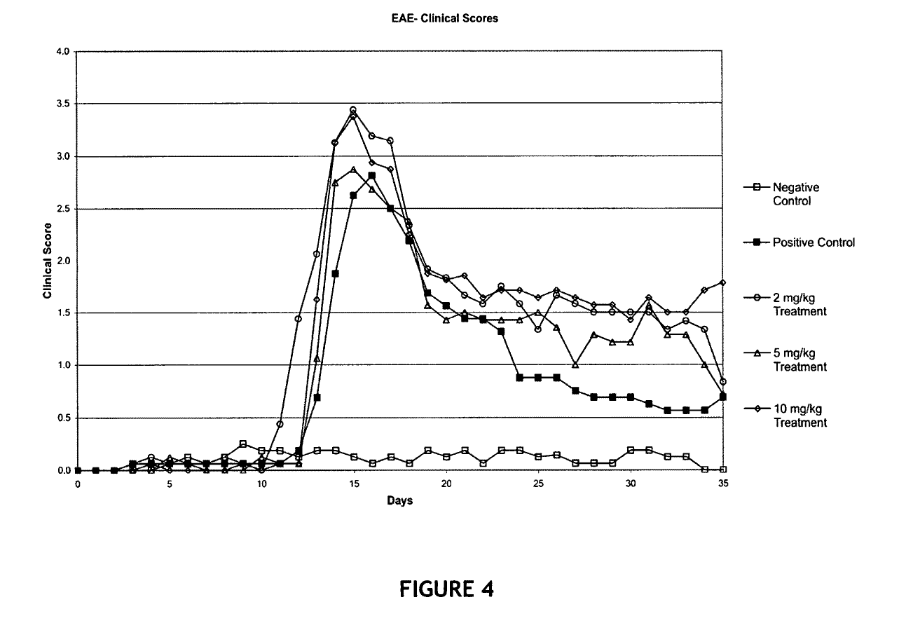

Both the body weights (FIG. 3) and clinical score (FIG. 4), in which the higher the number the worse the disease, show both an increase in the rate of onset and worsening of the disease on daily dosing of the Hsp inhibitor. The positive control group received only the vehicle and the negative control group was not immunized with the disease. The symbols represent the average of eight animals.

### Example 4

### Single vs. Weekly Dosing in MS Model

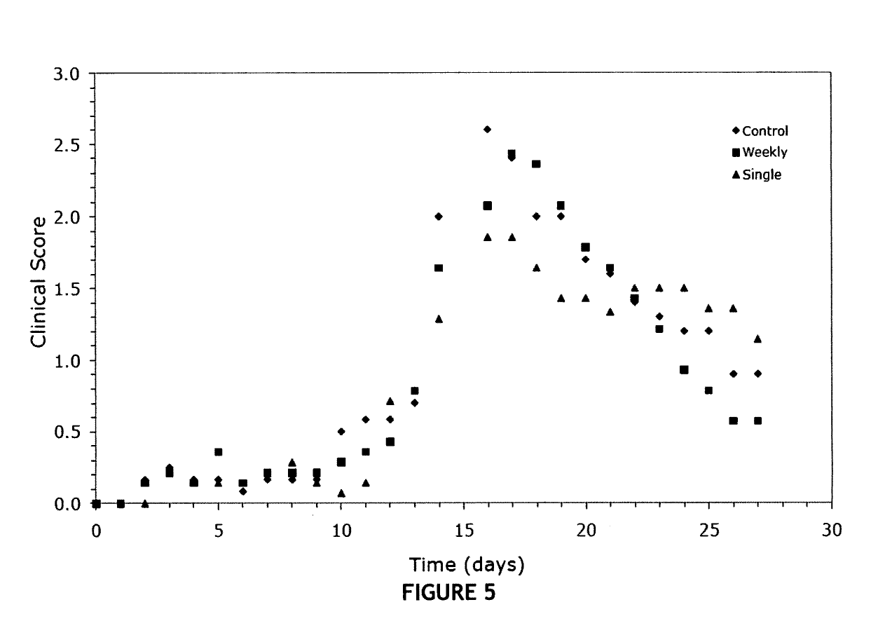

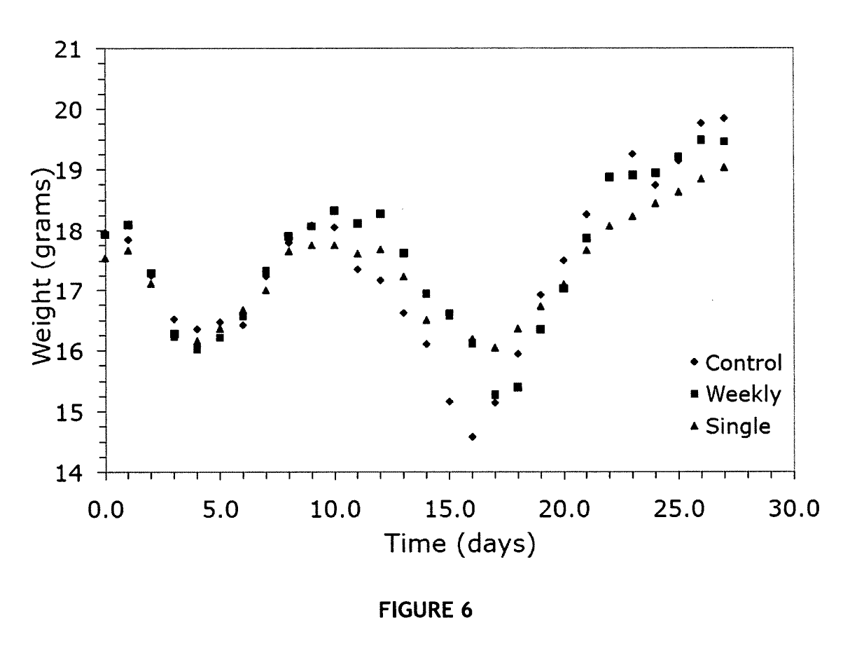

The effect of single versus multiple injections of Hsp inhibitors may be seen in the following MS data generated in a murine model. In this example, female SJL/J mice (6-7 weeks old) were immunized with proteolipid protein (“PLP”) in CFA and pertussis toxin IV on day 0. The mice were boosted with PLP in IFA on day seven. At the onset of symptoms, mice received either a single 20 mg/kg dose of KU-32 IP, weekly 2 mg/kg IP doses of KU-32, or no treatment. The mice were scored daily for severity of disease and body weight. As shown in the figures, the clinical score (FIG. 5) and body weight (FIG. 6), animals receiving the single injection had better clinical score and lost less body weight during the first onset of disease (10-20 days).

### Example 5

### HSP90 Inhibitor Efficacy in Reversing Sensory Nerve Conduction Deficits and Clinically Relevant Sensory Dysfunction

Diabetic peripheral neuropathy (DPN) results from the degeneration of nerves that transmit sensations from the legs and arms. Schwann cells are specialized cells that closely associate with many nerves and also undergo profound changes in DPN. Schwann cells are necessary to form the myelin membrane that is present on many peripheral nerves and the loss of this myelin membrane is a common characteristic of human diabetic neuropathy. Identifying molecular mechanisms that may prevent demyelination or enhance remyelination are therefore good targets for therapeutic interventions in human DPN. Altered neurotrophism in DPN is described in detail in, for example, Dobrowsky et al., JPET 313:485-491, 2005. Heat shock protein 90 inhibitors offer a potential therapeutic approach toward treating DPN. A cell culture model of myelinated nerve was used to identify new molecular targets that can prevent demyelination. KU-32 was found to prevent neuregulin-induced demyelination in a cell culture model.

Physiological studies were performed in a diabetic mouse model to determine if KU-32 is suitable for further pre-clinical development. A mouse model of streptozotocin (STZ)-induced diabetic neuropathy was utilized. One such diabetic mouse model is described, for example, in Christianson et al., Neuroscience, 2007, 145 (1): 303-313. Initial studies showed that after 8 weeks of diabetes, weekly administration of 20 mg/kg KU-32 by intraperitoneal injection was sufficient to improve thermal hypoalgesia and also helped to improve the decrease in sensory nerve conduction velocity (SNCV) that was observed after 15 weeks of diabetes. However, diabetes did not significantly decrease SNCV after 8 weeks which was when the KU-32 administration began. Thus, although the drug was capable of attenuating the decline in SNCV, a more powerful indicator of efficacy and clinical usefulness was desired to indicate the drug could reverse a pre-existing deficit in NCV. Additionally, it was noted that KU-32 seemed to have an effect on mechanical hypoalgesia and improved the pre-existing motor NCV (MNCV) deficit, but variability in a few animals in the small trail size negated it reaching significance. The study was therefore repeated with a larger trial size and under more stringent long-term diabetic conditions prior to weekly drug administration.

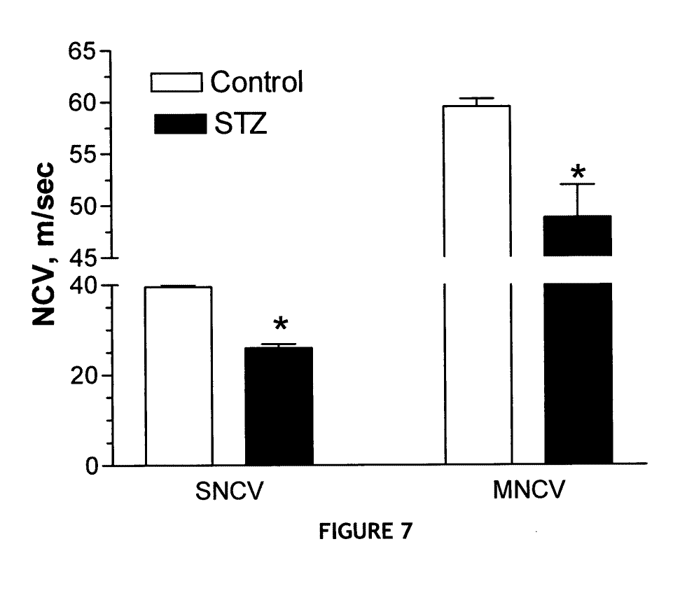

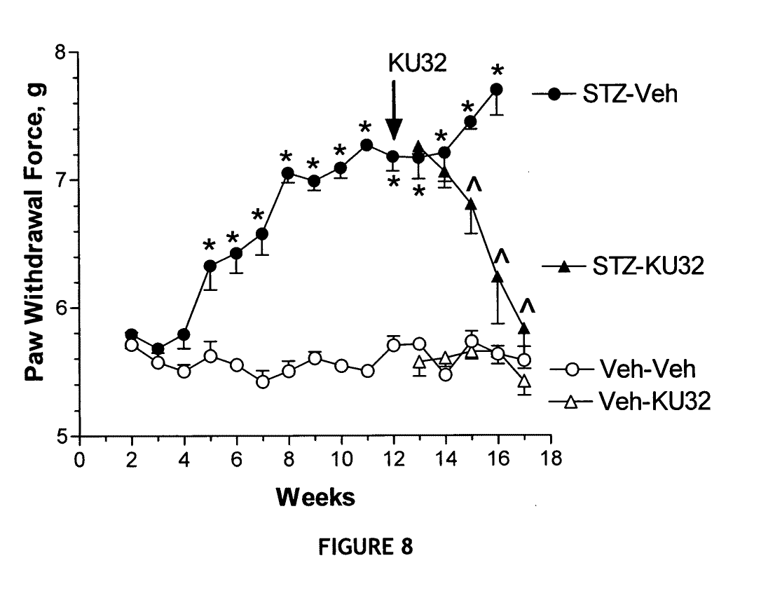

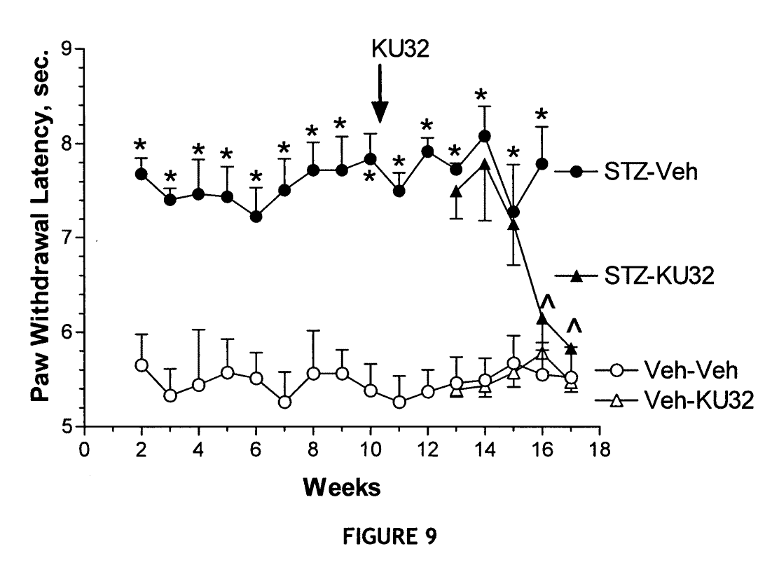

In a second trial, mice were rendered diabetic for 12 weeks. Diabetes was induced in test mice by 3 injections of streptozotocin (STZ) (n=28), and control mice (n=28) received a vehicle injection. Nerve conduction velocity (NCV) was measured in 4 mice from each group after 12 weeks, prior to initiating KU-32 treatment, after which time they developed substantial deficits in MNCV and SNCV (FIG. 7). Asterisks indicates p<0.05 compared to the time matched control. Diabetic mice also developed a substantial mechanical hypoalgesia after 5 weeks (FIG. 8) and a rapid onset of a thermal hypoalgesia (FIG. 9). Thus, any improvements by KU-32 were considered to be indicative of reversibility of a pre-existing deficits and not prevention. This is a critical clinical parameter as most patients do not seek medical therapy until the clear manifestation of sensory deficits which precede asymptomatic nerve conduction deficits.

After 12 weeks of diabetes, mice received weekly doses of 20 mg/kg KU-32 or vehicle by IP injection, and thermal and mechanical hypoalgesia were monitored. KU-32 significantly improved the mechanical hypoalgesia after 3 weeks of treatment and this continued to improve out to 5 weeks of treatment, as shown in FIG. 8. The asterisk indicates p<0.05 compared to the time matched control. The carat indicates p<0.05 compared to 14 weeks of STZ-Veh. Animal numbers were n=28 for Veh-Veh and STZ-Veh up to 12 weeks. Group numbers were n=12 at initiation of KU-32 and its vehicle (Veh). These data indicate that KU-32 can improve the function of more heavily myelinated fibers that transmit mechanical sensation.

Consistent with the prior results, KU-32 also improved thermal hypoalgesia which is mediated by thinly or unmyelinated fibers that undergo substantial damage in individuals with diabetic neuropathy, as shown in FIG. 9. The asterisk indicates p<0.05 compared to the time matched control, and the carat indicates p<0.05 compared to 14 weeks of STZ-Veh. Animal numbers were n=28 for Veh-Veh and STZ-Veh up to 12 weeks. Group numbers were n=12 at initiation of KU-32 and its vehicle (Veh).

Interestingly, when KU32 treatment was initiated after 8 weeks of diabetes in the initial studies, the improvement in thermal sensitivity was evident within two weeks. Importantly, a similar recovery was evident after 12 weeks of diabetes despite the onset in improvement requiring 4 weeks of treatment. The delay may relate to the more severe decline in the function of the smaller sensory fibers since treatment was not started until 12 weeks of diabetes. The diabetic mice were very ill after 16 weeks and therefore were terminated at this time. As can be seen in FIGS. S2 and S3, their responses were becoming much more variable due to their declining health. Since the deficits in mechanical and thermal hypoalgesia reached a plateau between 12-14 weeks, the measures at 14 weeks of diabetes were used to assess for statistical improvements in sensory function after 3 or more weeks of KU-32 treatment. On the other hand, the diabetic animals receiving KU-32 appeared more robust; despite no differences in fasting blood glucose or body weight compared to the diabetic mice. Thus, KU-32 improves nerve function in the absence of an overall improvement in metabolic parameters related to diabetes, i.e., hyperglycemia and weight loss. The data provides solid proof-of-principle that KU-32 can effectively reverse pre-existing deficits in two sensory measures that are direct, clinically relevant, parallels of human symptoms.

Since many possible embodiments may be made of the invention without departing from the scope thereof, it is to be understood that all matters herein set forth or shown in the accompanying drawings are to be interpreted as illustrative, and not in a limiting sense. While specific embodiments have been shown and discussed, various modifications may of course be made, and the invention is not limited to the specific forms or arrangement of parts and steps described herein, except insofar as such limitations are included in the following claims. Further, it will be understood that certain features and subcombinations are of utility and may be employed without reference to other features and subcombinations. This is contemplated by and is within the scope of the claims.

What is claimed is:

1. A method for treating or preventing a medical condition selected from the group consisting of a neurodegenerative disorder, an autoimmune disorder, and cancer, the method comprising administering according to a dosing regimen one or more doses of a therapeutically effective amount of at least one heat shock protein 90 inhibitor wherein said dosing regimen consists of no more than a single dose within a period of about 7 days.

2. The method of claim 1 wherein said dosing regimen consists of a single dose.

3. The method of claim 1 wherein said dosing regimen comprises at least two doses wherein the interval between consecutive doses is independently not less than about 7 days in length.

4. The method of claim 3 wherein the intervals between consecutive doses are each independently from about 7 days to about 28 days in length.

5. The method of claim 3 wherein the intervals between consecutive doses are each independently from about 14 days to about 28 days in length.

6. The method of claim 3 wherein the intervals between consecutive doses are each independently from about 7 days to about 14 days in length.

7. The method of claim 3 wherein the intervals between consecutive doses are each about 7 days in length.

8. The method of claim 3 wherein the intervals between consecutive doses are each 7 days in length.

9. The method of claim 3 wherein the intervals between consecutive doses are the same.

10. The method of claim 1 wherein said dose comprises from about 2 mg/kg to about 20 mg/kg of said heat shock protein 90 inhibitor.

11. The method of claim 3 wherein each said dose independently comprises from about 2 mg/kg to about 20 mg/kg of said heat shock protein 90 inhibitor.

12. The method of claim 1, wherein the heat shock protein 90 inhibitor is administered intravenously, intraperitoneally, or orally.

13. The method of claim 1 wherein said medical condition is cancer.

14. The method of claim 1 wherein said medical condition is an autoimmune disorder.

15. The method of claim 14 wherein said autoimmune disorder is multiple sclerosis.

16. The method of claim 1 wherein said medical condition is a neurodegenerative disorder.

17. The method of claim 16 wherein said neurodegenerative disorder is diabetic peripheral neuropathy.

18. The method of claim 1 wherein said Hsp90 inhibitor is selected from the group consisting of:
N-(7-((2R,3R,4S,5R)-3,4-dihydroxy-5-methoxy-6,6-dimethyl-tetrahydro-2H-pyran-2-yloxy)-2-oxo-2H-chromen-3-yl)acetamide;
N-(7-((2R,3R,4S,5R)-3,4-dihydroxy-5-methoxy-6,6-dimethyltetrahydro-2H-pyran-2-yloxy)-8-methyl-2-oxo-2H-chromen-3-yl)acetamide;
N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)-1H-indole-2-carboxamide;
N-(7-((2R,3R,4S,5R)-3,4-Dihydroxy-5-methoxy-6,6-dimethyl-tetrahydro-2H-pyran-2-yloxy)quinolin-3-yl)-4-methoxy-3-(3-methoxyphenyl)-benzamide;
3-(3′,6-dimethoxybiphenyl-3-ylcarboxamido)-8-methyl-2-oxo-2H-chromen-7-yl propionate;
3-(3′,6-dimethoxybiphenyl-3-ylcarboxamido)-8-methyl-2-oxo-2H-chromen-7-yl cyclopropane carboxylate; and
3-(3′,6-dimethoxybiphenyl-3-ylcarboxamido)-6-methoxy-8-methyl-2-oxo-2H-chromen-7-yl acetate.

19. The method of claim 1 wherein said Hsp90 inhibitor is selected from the group consisting of:
N-(7-((2R,3R,4S,5R)-3,4-dihydroxy-5-methoxy-6,6-dimethyl-tetrahydro-2H-py-ran-2yloxy)-2-oxo-2H-chromen-3-yl)acetamide; and
N-(7-((2R,3R,4S,5R)-3,4-dihydroxy-5-methoxy-6,6-dimethyltetrahydro-2H-pyran-2-yloxy)-8-methyl-2-oxo-2H-chromen-3-yl)acetamide.

20. A method for treating or preventing a medical condition selected from the group consisting of a neurodegenerative disorder, an autoimmune disorder, and cancer, the method comprising administering according to a dosing regimen n consecutive doses of a therapeutically effective amount of at least one heat shock protein 90 inhibitor to a subject in need thereof;
wherein said dosing regimen comprises n-1 intervals between said n consecutive doses;
wherein each of said n-1 intervals is independently not less than about 7 days in length;
and where n is at least two.

21. The method of claim 20 wherein said medical condition is multiple sclerosis.

22. The method of claim 20 wherein said medical condition is diabetic peripheral neuropathy.

23. The method of claim 20 wherein said Hsp90 inhibitor is selected from the group consisting of:
N-(7-((2R,3R,4S,5R)-3,4-dihydroxy-5-methoxy-6,6-dimethyl-tetrahydro-2H-pyran-2-yloxy)-2-oxo-2H-chromen-3-yl)acetamide;
N-(7-((2R,3R,4S,5R)-3,4-dihydroxy-5-methoxy-6,6-dimethyltetrahydro-2H-pyran-2-yloxy)-8-methyl-2-oxo-2H-chromen-3-yl)acetamide;
N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)-1H-indole-2-carboxamide;
N-(7-((2R,3R,4S,5R)-3,4-Dihydroxy-5-methoxy-6,6-dimethyl-tetrahydro-2H-pyran-2-yloxy)quinolin-3-yl)-4-methoxy-3-(3-methoxyphenyl)-benzamide;
3-(3′,6-dimethoxybiphenyl-3-ylcarboxamido)-8-methyl-2-oxo-2H-chromen-7-yl propionate;
3-(3′,6-dimethoxybiphenyl-3-ylcarboxamido)-8-methyl-2-oxo-2H-chromen-7-yl cyclopropane carboxylate; and
3-(3′,6-dimethoxybiphenyl-3-ylcarboxamido)-6-methoxy-8-methyl-2-oxo-2H-chromen-7-yl acetate.

24. The method of claim 20 wherein said Hsp90 inhibitor is selected from the group consisting of:
N-(7-((2R,3R,4S,5R)-3,4-dihydroxy-5-methoxy-6,6-dimethyl-tetrahydro-2H-py-ran-2-yloxy)-2-oxo-2H-chromen-3-yl)acetamide; and
N-(7-((2R,3R,4S,5R)-3,4-dihydroxy-5-methoxy-6,6-dimethyltetrahydro-2H-pyran-2-yloxy)-8-methyl-2-oxo-2H-chromen-3-yl)acetamide.

25. The method of claim 20 wherein each of said n-1 intervals is independently about 7 days to about 28 days in length.

26. The method of claim 20 wherein each of said n-1 intervals is independently about 7 days to about 14 days in length.

27. The method of claim 20 wherein each of said n-1 intervals is independently about 14 days to about 28 days in length.

28. A method for treating or preventing a medical condition selected from the group consisting of multiple sclerosis and diabetic peripheral neuropathy, the method comprising:
administering a plurality of consecutive doses of a therapeutically effective amount of at least one heat shock protein 90 inhibitor to a subject in need thereof, wherein the interval between consecutive doses is independently from about 7 days to about 28 days in length,
and wherein said at least one Hsp90 inhibitor is selected from the group consisting of
N-(7-((2R,3R,4S,5R)-3,4-dihydroxy-5-methoxy-6,6-dimethyl-tetrahydro-2H-py-ran-2-yloxy)-2-oxo-2H-chromen-3-yl)acetamide; and
N-(7-((2R,3R,4S,5R)-3,4-dihydroxy-5-methoxy-6,6-dimethyltetrahydro-2H-pyran-2-yloxy)-8-methyl-2-oxo-2H-chromen-3-yl)acetamide.

29. The method of claim 28 wherein each dose is in the form of a pharmacetical composition comprising said at least one Hsp 90 inhibitor and further comprising at least one pharmaceutically acceptable carrier.
